(19) United States
(12) Patent Application Publication
(10) Pub. No.: US 2024/0009297 A1
(43) Pub. Date: Jan. 11, 2024

(54) Antibody/Adjuvant Compositions and Methods of Immune Response Generation against Coronaviruses

(71) Applicants: Soligenix, Inc., Princeton, NJ (US); University of Hawaii, Honolulu, HI (US)

(72) Inventors: Oreola Donini, Coquitlam (CA); Axel Lehrer, Honolulu, HI (US)

(21) Appl. No.: 18/017,752

(22) PCT Filed: Jul. 26, 2021
(86) PCT No.: PCT/US21/43236

§ 371 Date: Jan. 24, 2023

Related U.S. Application Data
(60) Provisional application No. 63/156,200, filed on Mar. 3, 2021.
(60) Provisional application No. 63/056,385, filed on Jul. 24, 2020.

(51) Int. Cl. A61K 39/215; A61K 39/39; A61P 31/14

(52) U.S. Cl. CPC A61K 39/215; A61K 39/39; A61P 31/14; A61K 2039/55583

(57) ABSTRACT

Recombinant vaccine compositions combined with a specified adjuvant to deliver a robust immune response against coronaviruses for inducing protective immunity to a coronavirus comprising providing a stable immunogenic composition capable of eliciting a robust and durable immune response to the coronavirus, wherein the composition comprises a protein subunit comprising a recombinant protein specific to the coronavirus and at least one adjuvant.

### RELATED APPLICATIONS

This application is a national stage application under 35 U.S.C. 371 claiming priority from International Patent Application No. PCT/US21/43236 filed Jul. 26, 2021, which claims the benefit of priority from U.S. Provisional Application No. 63/156,200 filed Mar. 3, 2021, and U.S. Provisional Application No. 63/056,385 filed Jul. 24, 2020, the contents of which are hereby incorporated by reference herein.

### FIELD OF THE INVENTION

The invention relates to the field of recombinant vaccine compositions combined with a specified adjuvant to deliver a robust immune response against coronaviruses.

### BACKGROUND OF THE INVENTION

The emergence and rapid spread of a new infectious respiratory disease, Coronavirus Disease 2019 (COVID-19) has caused an unprecedented public health emergency worldwide since emerging in December 2019. A novel coronavirus, severe acute respiratory syndrome coronavirus 2 (SARS-CoV-2), which is closely related to SARS-CoV, was identified as the etiologic agent of the new respiratory disease. Despite the practice of public health strategies such as social distancing and use of face masks, numbers of COVID-19 cases continue to rise globally. There is an urgent need for safe and effective, inexpensive and easily deployed vaccines that would rapidly establish herd immunity to break transmission on a global scale.

Facilitated by the broad nature of the outbreak, significant SARS-CoV-2 variants have been identified, some of which appear to be more resistant to first-generation vaccines. More than 200 COVID-19 vaccine candidates using various technology platforms are currently in development [2]. Among these, two frontrunning vaccines based on mRNA platforms, Pfizer/BioNTech BNT162b2 and Moderna mRNA-1273 with reported interim efficacy rates of 95% and 94.1% respectively, have been approved by the U.S. Food and Drug Administration (FDA) for emergency use in mid-December 2020 [3, 4]. These two vaccines are now being administered in the U.S. and have also been approved for use in other jurisdictions with most authorities recommending administration to high-risk individuals only as supply is limited. Furthermore, both vaccines require stringent cold-chain distribution and storage.

For the furthest advanced vaccines, at least one booster dose seems necessary, bringing global demand to at least 16 billion doses [5]. While the recent Emergency Use Authorization (EUA) approval of the Janssen Ad26.COV2.S vaccine, which showed 85% efficacy in preventing severe COVID-19, is expected to ease this burden [6, 7], no single vaccine can be produced rapidly in sufficient quantities to satisfy the global urgent need, diversification using different vaccine platforms would enable worldwide vaccine coverage as well as address the needs of the most vulnerable populations, particularly elderly and immunocompromised individuals or those with other co-morbidities. With mutations emerging even in the absence of significant selection pressure placed on SARS-CoV-2, the initiation of vaccination programs may further enhance strain diversity. Thus, continued research into adaptable and more easily distributed vaccines, compatible with rapid deployment and significant cost efficiencies, must continue unabated.

The recombinant subunit vaccine platform offers a safety advantage over virally vectored vaccines and a distribution advantage relative to many other vaccine platforms. Purified recombinant protein antigens can be engineered to achieve optimal immunogenicity and protective efficacy. Furthermore, a thermostabilized subunit vaccine can be deployed in the field, eliminating stringent cold-chain requirements. Formulation of the vaccine immunogen with a potent adjuvant enhances and focuses immunogenicity while lowering the antigen dose requirement, thereby enabling vaccination of more people with a product carrying significantly more clinical and regulatory precedence compared to nucleic acid-based approaches.

CoVaccine HT™, an oil-in-water nanoemulsion adjuvant with excellent safety, immunogenicity and stability, in combination with properly selected antigens can achieve potent immunogenicity and protective efficacy in rodents and non-human primates (NHPs) [8]. Previous work has successfully demonstrated the use of recombinant protein subunit Drosophila S2 expression system in combination with CoVaccine HT™ to produce vaccines to combat global health threats such as Zika virus (ZIKV) and Ebola virus (EBOV). Immunization with recombinant ZIKV E protein induced potent neutralizing titers in mice [9] and non-human primates [8] and protection against viremia after viral challenge. Similarly, immunization with recombinant subunit formulations consisting of the EBOV glycoprotein and matrix proteins VP40 and VP24 was able to induce potent antibody titers and protection in both mouse and guinea pig models [11].

More recently, we have demonstrated that these recombinant subunits, in combination with CoVaccine HT™, can be formulated as a glassy solid using lyophilization. In this format, lyophilized vaccine formulations consisting of filovirus glycoprotein and adjuvant were able to maintain native quaternary structure and immunogenicity for at least 12 weeks at 40° C. [12, 13]. Other vaccines, using a similar lyophilization process, demonstrated potency after at least 1 year at 40° C. [14]. Thus, we have the capacity to produce a lyophilized, adjuvanted recombinant protein vaccine in a single vial, which is stable for long term storage even at elevated temperatures and which can be reconstituted with sterile water for injection immediately prior to use.

Spike (S) glycoprotein, comprised of a receptor binding subunit (51) and a membrane-fusing subunit (S2) is the main surface protein and present as homotrimers on the viral envelope of SARS-CoV-2. Based on previous preclinical studies of vaccines against the highly pathogenic SARS-CoV and Middle East respiratory syndrome coronavirus (MERS-CoV) [16-18] as well as recent studies of patients with SARS-CoV-2 infections [19-22], S protein appears to be the antigenic target of both neutralizing antibody and T cell responses. The majority of current COVID-19 vaccines under preclinical and clinical development use full-length S proteins as antigen targets with further modifications such as removal of the polybasic sites [23-25], introduction of proline mutations [23, 26, 27], or addition of trimerization domains to preserve the native-like trimeric prefusion structure of S proteins. These antigens have been shown to mimic the native S protein presented on viral particles and preserve neutralization-sensitive epitopes [18, 28]. In a prior study, we evaluated the utility of CoVaccine HT™ adjuvant to induce properly balanced immunity against SARS-CoV-2, when formulated with a commercially available SARS- CoV-2 spike S1 protein. This work demonstrated that CoVaccine HT™ is an effective adjuvant that promotes rapid induction of balanced humoral and cellular immune responses [29]. However, no consensus is established on an optimal adjuvant system, including specific dosing requirements, that best induces protective immunity to SARS-CoV-2.

### SUMMARY OF THE INVENTION

The present invention provides for a method of inducing protective immunity to a coronavirus comprising providing a stable immunogenic composition capable of eliciting a robust and durable immune response to the coronavirus, wherein the composition comprises a protein subunit comprising a recombinant protein specific to the coronavirus and at least one adjuvant; and administering an effective amount of the composition to the individual. Preferably, the recombinant protein specific to the coronavirus is expressed using insect cells. Optionally, the protein subunit is a spike protein from the coronavirus. More preferably, the spike protein is further purified using immunoaffinity purification.

In another aspect, the protein subunit is present in the composition from about 1 μg to about 25 μg. Preferably, the at least one adjuvant is a sucrose fatty acid sulphate ester. Most preferably, the sucrose fatty acid sulphate ester is CoVaccine HT™. Optionally, the recombinant protein is included in a lower quantity than the at least one adjuvant. More preferably, the at least one adjuvant is in an amount from about 0.3 mg to about 10 mg within the composition for administration to the individual.

In yet another aspect, the protein subunit and the at least one adjuvant are each thermostabilized separately before being combined in the composition. Optionally, the protein subunit and the at least one adjuvant are thermostabilized together before being combined in the composition.

In an alternative embodiment, the present invention provides for a method of adjuvanting subunit vaccines, comprising providing an effective amount of an adjuvant, providing a protein subunit and combining the adjuvant and the protein subunit to create a stable immunogenic composition capable of eliciting a robust and durable immune response to a coronavirus, wherein the adjuvant is a sucrose fatty acid sulphate ester at a concentration selected from the group consisting of between 0.3 and 1 mg, 1 mg and 5 mg and 5 mg and 10 mg. Preferably, the recombinant protein specific to the coronavirus is expressed using insect cells. Optionally, the protein subunit is a spike protein from the coronavirus. More preferably, the spike protein is further purified using immunoaffinity purification.

In another aspect, the protein subunit is present in the composition from about 1 μg to about 25 μg. Optionally, the recombinant protein is included in a lower quantity than the at least one adjuvant.

In yet another aspect, the protein subunit and the at least one adjuvant are each thermostabilized separately before being combined in the composition. Optionally, the protein subunit and the at least one adjuvant are thermostabilized together before being combined in the composition.

### BRIEF DESCRIPTION OF THE DRAWINGS

The following figures provide illustrative examples of the present invention and are incorporated by reference within this disclosure.

FIG. 1 depicts immunogenicity and specificity to SARS-CoV-2 S1 immunization. A: Timeline schematic of BALB/c immunizations and bleeds with a table detailing the study design. B: Median fluorescence intensity (MFI) of serum antibodies from each group binding to custom magnetic beads coupled with Spike S1 proteins from either SARS-CoV-2 (SARS-2), SARS-CoV (SARS), or MERS-CoV (MERS) on day 14 and 35. C: Antibody reactivity to SARS-2, SARS, and MERS antigens throughout the study. Graphs in panels B and C are on a logarithmic scale representing geometric mean MFI responses with 95% confidence interval (CI). The dashed lines represent assay cut-off values determined by the mean plus three standard deviations of the negative control (BSA coupled beads).

FIG. 2 shows serum IgG titres against Coronavirus S1 antigens. A: Antigen reactivity in a four-fold dilution series of mouse sera. B: Area under the curve (AUC) of data in A. Both graphs are in log scale with geometric mean and 95% CI. The dashed lines in panel A represent the cut-off value determined by the mean plus three standard deviations of the negative control (BSA coupled beads). K=x1000, M=x1,000,000, D.F.=dilution factor. Statistics by standard one way-ANOVA. **** =p<0.0001.

FIG. 3 shows adjuvant effects on immunoglobulin subclass diversity. A: IgG subclasses reacting with SARS-2-S1 antigen between day 14 and day 35 plotted on a linear scale. B: Relative abundance of Immunoglobulin isotypes and IgG subclasses reacting to SARS-2 and SARS antigens determined by subtracting the specified subclass cut-off values from the geometric mean of each group. The total MFI from which the subclasses are a fraction of is listed below each pie-chart. C: Ratios of subclasses. The normalized MFI values of each subclass per mouse were plotted as ratios using geometric mean and 95% CI.

FIG. 4 shows detection of IFN-y-secreting cells from mice immunized with SARS-CoV-2 vaccines. The splenocytes were obtained from mice (2 to 3 per group) immunized with SARS-CoV-2 S1 protein, adjuvanted with CoVaccine HT™ or Alum, or S1 protein alone on day 28 (one week after booster immunizations). Pooled splenocytes obtained from two naïve mice were used as controls. The cells were incubated for 40 hours with PepTivator ® SARS-CoV-2 Prot_S1 peptide pools at 0.2 μg/mL or 0.5 μg/mL per peptide or medium. IFN-γ secreting cells were enumerated by FluoroSpot. The results are expressed as the number of spot forming cells (SFC) after subtraction of the number of spots formed by cells in medium only wells to correct for background activity. *** p≤0.001, **** p≤0.0001.

FIG. 5 depicts IgG antibody responses to recombinant SARS-CoV-2 S proteins. (A) Groups of Swiss Webster mice (n=7 or 15) were immunized with one or two doses of recombinant S proteins with or without CoVaccine HT™ (CoVac) adjuvant at a 3-week interval. Sera were collected 1 and 2 weeks after each immunization (days 7, 14, 28, and 35), and spleens were harvested 1 week (day 7 and 28) after each immunization. SARS-CoV-2 S-specific IgG titers were measured by a multiplex microsphere immunoassay (MIA) using SdTM2P and RBD-F coupled beads. (B) The purified anti-S antibody was diluted to concentrations in the range of 4.8 to 5000 ng/mL and analyzed by MIA as a standard (as described in the Materials and Methods). Mouse sera were assayed along with the antibody standard and the IgG concentrations were interpolated from the standard curves using a sigmoidal dose-response computer model (GraphPad Prism). The dotted lines denote the top and bottom of linear range that were used to interpolate antibody concentration (C) The anti-S and (D) anti-RBD antibody titers in sera from mice immunized with SdTM or SdTM2P (purified by hACE2 AC) with or without adjuvants or (E) anti-S antibody in sera of mice administered different dosages (5, 2.5, or 1.25 μg) of SdTM2P (purified by mAb IAC) with adjuvant (1 or 0.3 mg) are expressed as IgG concentrations (ng/mL). The dotted lines in panels C to E indicate the bottom of linear range of the standard curve.

FIG. 6 shows serum neutralization titers of mice immunized with recombinant subunit SARS-CoV-2 vaccines. The neutralization titers of sera obtained 2 weeks post boosting (day 35) from mice vaccinated with (A) SARS-CoV-2 SdTM or SdTM2P proteins (purified by hACE2 AC) adjuvanted with or without 1 mg of CoVaccine HT™ (CoVac) or (B) different dosages (5, 2.5, or 1.25 μg) of SdTM2P (purified by mAb IAC) with CoVac (1 or 0.3 mg) were measured by a PRNT using rVSV-SARS-CoV-2-S. The data shown are log-transformed PRNT50 values from individual animals, and mean ±SEM of each group is indicated. Horizontal dashed lines represent the limit of detection. For the samples showing low (<50%) or no neutralizing activity at the starting dilution (1:10) in the assay, a PRNT₅₀ of 5 (half of the limit of detection) was reported when the data were plotted. One sample t test on log-transformed data was used to analyze the significant difference between adjuvanted and protein alone groups in panel A (*p<0.05, **p<0.01). There are no significant differences between groups when analyzed by one-way ANOVA with Tukey' s multiple comparisons test in panel B. (C) The correlation between the neutralization titers of pooled serum samples from the same group to rVSV-SARS-CoV-2 and those to WT SARS-SARS-CoV-2 was examined by Pearson correlation analysis.

FIG. 7 shows IgG subclass profile induced after vaccination with recombinant S proteins with or without CoVaccine HT™ (CoVac) adjuvant. (A) Mouse sera collected at two weeks post-booster (day 35) were assessed for anti-S specific IgG1, IgG2a, and IgG2b by MIA. The ratios of IgG2a to IgG1 (B) or IgG2b to IgG1 (C) from individual animals were calculated using the MFI values at the serum dilution of 1:2,000. Bars represent mean ±SEM of each group. Significant difference in the ratios between adjuvanted and protein alone groups was determined by Mann-Whitney t test (*p<0.05).

FIG. 8 shows Detection of IFN-γ secreting cells from mice immunized with SARS-CoV-2 vaccines. The splenocytes were obtained from mice (n=3 or 5 per group) immunized with one dose (open symbols) or two doses (closed symbols) of (A) either 5 μg of SARS-CoV-2 SdTM or SdTM2P proteins (purified by hACE2 AC) adjuvanted with 1 mg of CoVaccine HT™ (CoVac) or (B) different dosages (5, 2.5, or 1.25 μg) of SdTM2P (purified by mAb IAC) with CoVac (1 or 0.3 mg). The cells were incubated for 24 hours with medium or a peptide pool covering the S protein of SARS-CoV-2 (10 μg/mL), and the IFN-γ secreting cells were enumerated by FluoroSpot. The data are shown as the mean values of triplicate assays from individual animals using the number of spot forming cells (SFC) per 10⁶ splenocytes after subtraction of the number of spots formed by cells in medium control wells (<5 spots). Bars represent mean ±SEM of each group. Significant differences in numbers of IFN-γ secreting cells between groups receiving 2 doses of vaccines with or without adjuvant in panel A was determined by Mann-Whitney t test (*p<0.05). There are no significant differences between groups given either one or two doses of vaccines when analyzed by one-way ANOVA with Tukey's multiple comparisons test in panel B.

FIG. 9 shows PRNT₅₀ (A) and PRNT90 (B) neutralizing antibody levels obtained in non-naïve cynomolgus macaques (N=3/group) after vaccination on Days 0 and 21, with blood collection on Day 35. A robust antibody response was generated with 5 mg of CoVaccine HT™ used as either the liquid or lyophilized formulation.

FIG. 10 shows groups of Cynomolgus Macaques (n=3) were immunized with two doses of the SARS CoV2 spike protein formulated with either liquid or lyophilized CoVaccine HT™ in 3-week intervals. Sera was collected at weeks 0, 3, 5, 6 and 15. NHPs were challenged with the P.1 Gamma Variant of Concern at week 15.

FIG. 11 shows the anti-S antibody titers in sera from NHPs immunized with each vaccine formulation. See FIG. 10 for legend.

FIG. 12 shows the anti-RBD antibody titers in sera from NHPs immunized with each vaccine formulation. See FIG. 10 for legend.

FIG. 13 shows neutralizing antibody titers calculate using a PRNT using WT-SARS-CoV-2, A, USA-WA1/2020 strain. Statistical differences of IgG and PRNT₅₀ between groups were calculated using a two-way ANOVA followed by a Tukey's Multiple Comparison. p-value≤0.05, ** p-value≤0.01, *** p-value≤0.001. See FIG. 10 for legend.

FIG. 14 shows the anti-S1 antibody titers against the B1.1.1.7 Alpha in sera from NHPs immunized with each vaccine formulation. Statistical differences of IgG titers between groups were calculated using a two-way ANOVA followed by a Tukey's Multiple Comparison. See FIG. 10 for legend.

FIG. 15 shows the anti-S1 antibody titers against the B.1.351 Beta S1 protein in sera from NHPs immunized with each vaccine formulation. Statistical differences of IgG titers between groups were calculated using a two-way ANOVA followed by a Tukey's Multiple Comparison. See FIG. 10 for legend.

FIG. 16 shows inverse correlation between (A) anti-S, (B) anti-RBD, (C) USA-WA1/2020 SARS-CoV-2 WT PRNT₅₀ and (D) rVSV-SARS CoV2 PRNT₅₀ at week 15 to TCID₅₀ from bronchial alveolar lavage (BAL). Correlation was calculated using a two-tail Spearman's correlation test. See FIG. 10 for legend.

FIG. 17 shows inverse correlation between (A) anti-S, (B) anti-RBD, (C) USA-WA1/2020 SARS-CoV-2 WT PRNT₅₀ and (D) rVSV-SARS CoV2 PRNT₅₀ at week 15 nasal swab TCID₅₀ 2 days post-challenge. Correlation was calculated using a two-tail Spearman's correlation test. See FIG. 10 for legend.

### DETAILED DESCRIPTION OF THE INVENTION

In order to investigate which adjuvants induce a strong humoral response, protein subunit vaccine candidates were formulated using a recombinant SARS-CoV-2 spike protein, produced as described below, adjuvanted with CoVaccine HT™ (or Alhydrogel®) at varying concentrations and in different formats (liquid or lyophilized) in mice and non-human primates. The former is a proprietary adjuvant and the latter is an FDA approved adjuvant used in several FDA licensed vaccines. CoVaccine HT™ is an oil-in-water emulsion of hydrophobic, negatively-charged sucrose fatty acid sulphate esters with the addition of squalane (Stevens, N. E. et al. An empirical approach towards the efficient and optimal production of influenza-neutralizing ovine polyclonal antibodies demonstrates that the novel adjuvant CoVaccine HT™ is functionally superior to Freund's adjuvant. PLoS One 8, e68895, doi:10.1371/journal.pone.0068895 (2013); Blom, A. G. & Hilgers, L. A. Sucrose fatty acid sulphate esters as novel vaccine adjuvants: effect of the chemical composition. Vaccine 23, 743-754, doi :10.1016/j .vaccine.2004.07.021 (2004)). Alhydrogel® is a colloid of aluminum hydroxide which binds protein to facilitate antigen recognition and thus, improve the immune response (Harris, J. R. et al. Alhydrogel® adjuvant, ultrasonic dispersion and protein binding: a TEM and analytical study. Micron 43, 192-200, doi:10.1016/j.micron.2011.07.012 (2012)).

The mechanism of action of Alhydrogel remains somewhat elusive, however this adjuvant likely interacts with NOD like receptor protein 3 (NLRP3) but does not interact with TLRs (Sun, H., Pollock, K. G. & Brewer, J. M. Analysis of the role of vaccine adjuvants in modulating dendritic cell activation and antigen presentation in vitro. Vaccine 21, 849-855, doi:10.1016/s0264-410x(02)00531-5 (2003)). This difference in cellular activation can account for the disparities seen between the use of CoVaccine HT™ and Alhydrogel® presented here. The stabilized oil in water emulsion functions by generating a response skewed towards a Th1 direction which can in turn sustain CD8 T cells capable of mitigating viral infection (Snell, L. M. et al. Overcoming CD4 Th1 Cell Fate Restrictions to Sustain Antiviral CD8 T Cells and Control Persistent Virus Infection. Cell Rep 16, 3286-3296, doi :10.1016/j.celrep.2016.08.065 (2016)). This adjuvant is also capable of inducing T cell differentiation to Tfh cells which is evident through class switching to IgG2a. In concert, these cellular responses enhance the humoral response evidenced by the overall higher titres of IgG. CoVaccine HT™ also offers an advantage in comparison to Alhydrogel® regarding particle size. Alhydrogel® particles typically fall within the range of 1-10 microns (Orr, M. T. et al. Reprogramming the adjuvant properties of aluminum oxyhydroxide with nanoparticle technology. NPJ Vaccines 4, 1, doi:10.1038/s41541-018-0094-0 (2019) whereas Covaccine HT™ is typically less than 1 micron (Hilgers, L. A. T., Platenburg, P. L. I., Luitjens, A., Groenveld, B., Dazelle, T., Ferrari-Laloux, M., & Weststrate, M. W. . A novel non-mineral oil-based adjuvant. I. Efficacy of a synthetic sulfolipopolysaccharide in a squalane-in-water emulsion in laboratory animals. Vaccine 12, 653-660, doi:10.1016/0264-410x(94)90272- 0 (1994)). Smaller particle sizes offer increased stability and enhanced adjuvanticity. In summary, CoVaccine HT™ could provide a distinct advantage over Alhydrogel® as the more conventional adjuvant choice.

Here we demonstrate that the adjuvant Covaccine HT™ more rapidly induces humoral immunity characterized by high IgG titres, class-switched cross reactive and high neutralizing antibody titers with a stronger bias towards Th1 type responses and induces potent cell-mediated immunity responses which were absent in formulations containing protein alone or in combination with Alhydrogel®. The cross-reactive nature of the antibody responses (to the corresponding protein of SARS-CoV) indicates a potential for generating antibodies against conserved regions of the spike S1 domain. Most importantly, the high titres induced by CoVaccine HT™ after a single dose in conjunction with the observed class switching after two doses, suggest that partial to full protection of a vaccine adjuvanted with CoVaccine HT™ may be achieved sooner than if adjuvanted with Alhydrogel ® .

The present invention provides for a native-like, trimeric S protein ectodomain with and without stabilizing mutations using the Drosophila S2 cell expression system which was used to assess the immunogenicity of these S ectodomain trimers formulated with CoVaccine HT™ in mice and non-human primates.

It is generally accepted that vaccine dose equivalence between mice and non-human primates and humans is approximately 10-fold. Herein we use immunogenicity in non-human primates as a correlate for human dosing.

### EXAMPLES

The following examples illustrate the various embodiments of the present invention and are not meant to be limiting in scope based on such examples.

### Mouse Experiments with Prototype S1 Antigen and Methods

### I. Prototype Antigen: Vaccination and Serum Collection

BALB/c mice (7-8 weeks of age) were immunized twice, three weeks apart, intramuscularly (i.m.) with 5 μg of SARS-CoV-2 spike-S1 (Sino Biological 40592-VO5H) protein with or without adjuvants, or adjuvant alone, using an insulin syringe with a 29-gauge needle. The adjuvants used were CoVaccine HT™ (Protherics Medicines Development Ltd, London, United Kingdom), or 2% Alhydrogel® adjuvant (InvivoGen, San Diego, CA). Sera were collected by tail bleeding at 2 weeks post immunization or cardiac puncture for terminal bleeds. An additional serum sample was collected by cardiac puncture at day 28 along with splenocytes from three animals in the spike S1 +CoVaccine HT™ (S1+CoVac) and S1+Alum groups, and two animals in the S1+PBS group.

### II. Serological Immunoglobulin Assays

Internally dyed, carboxylated, magnetic microspheres (Mag-PlexTM-C) were obtained from Luminex Corporation (Austin, TX, USA). The coupling of individually addressable microspheres with all previously mentioned proteins were conducted as described previously (Namekar, M., Kumar, M., O'Connell, M. & Nerurkar, V. R. Effect of serum heat-inactivation and dilution on detection of anti-WNV antibodies in mice by West Nile virus E-protein microsphere immunoassay. PLoS One 7, e45851, doi:10.1371/journal.pone.0045851 (2012); Wong, S. J. et al. Detection of human anti-flavivirus antibodies with a west nile virus recombinant antigen microsphere immunoassay. J Clin Microbiol 42, 65-72 (2004)). Microspheres dyed with spectrally different fluorophores were also coupled with bovine serum albumin as a negative control. SARS-CoV-2, SARS-CoV, and MERS-CoV specific immunoglobulin antibody titres in mouse sera were measured using a microsphere immunoassay as previously described with some minor alterations (Haun, B. K. et al. Serological evidence of Ebola virus exposure in dogs from affected communities in Liberia: A preliminary report. PLoS neglected tropical diseases 13, e0007614, doi :10.1371/j ournal.pntd.0007614 (2019); Kumar, M., O'Connell, M., Namekar, M. & Nerurkar, V. R. Infection with non-lethal West Nile virus Eg101 strain induces immunity that protects mice against the lethal West Nile virus NY99 strain. Viruses 6, 2328-2339, doi:10.3390/v6062328 (2014); To, A. et al. Recombinant Zika Virus Subunits Are Immunogenic and Efficacious in Mice. mSphere 3, doi:10.1128/mSphere.00576-17 (2018)).

Briefly, microspheres coupled to his-tagged Spike-S1 proteins of SARS-CoV-2, SARS-CoV, or MERS-CoV (Sino Biological 40591-V08H, 40150-V08B1, & 40069-V08H, respectively), and control beads coupled to bovine serum albumin (BSA) were combined and diluted in MIA buffer (PBS-1% BSA-0.02% Tween20) at a dilution of 1/200. Multiplex beads (at 50 μL containing approximately 1,250 beads of each type) were added to each well of black-sided 96-well plates. 50 μL of diluted serum were added to the microspheres in duplicate and incubated for 3 hours on a plate shaker set at 700 rpm in the dark at 37° C. The plates were then washed twice with 200 μL of MIA buffer using a magnetic plate separator (Millipore Corp., Billerica, MA). 50 μL of red-phycoerythrin (R-PE) conjugated F(ab′)2 fragment goat anti-mouse IgG specific to the Fc fragment (Jackson ImmunoResearch, Inc., West Grove, PA) was added at 1 μg/ml to the wells and incubated for 45 minutes. Antigen-specific IgG subclass titres were determined using mouse antisera at a 1:1000 dilution. Detection antibodies were subclass specific goat anti-mouse polyclonal R-PE-conjugated antibodies (Southern Biotech) used at a 1:200 dilution. The plates were washed twice, as described above, and microspheres were then resuspended in 120 μl of drive fluid and analyzed on the MAGPIX Instrument (MilliporeSigma). Data acquisition detecting the median fluorescence intensity (MFI) was set to 50 beads per spectral region. Antigen-coupled beads were recognized and quantified based on their spectral signature and signal intensity, respectively. Assay cut-off values were calculated first by taking the mean of technical duplicate values using the average MFI (indicated as a dashed black line) from the adjuvant only control group. Cut-offs were generated by determining the mean MFI values plus three standard deviations as determined by Microsoft Office Excel program. Graphical representation of the data was done using Prism, Graphpad Software (San Diego, CA).

### III. Plaque Reduction Neutralization Test

A PRNT was performed in a biosafety level 3 facility (Bioqual) using 24-well plates. Mouse serum was pooled, diluted to 1:20, and a 1:3 serial dilution series was performed 11 times. Diluted samples were incubated with 30 plaque-forming units for 1 hr at 37° C. The serum-virus mixtures were added to a monolayer of confluent Vero E6 cells and incubated for 1 hour at 37° C. in 5% CO₂. Each well was then overlaid with 1mL of 0.5% methylcellulose media and incubated for 3 days. The plates were then fixed with methanol at −20° C. for 30 minutes and stained with 0.2% crystal violet for 30 minutes at room temperature. Neutralization titres were defined as the highest serum dilution resulted in 50% (PRNT50) and 90% (PRNT90) reduction in the number of plaques.

### IV. Preparation of Mouse Splenocytes and FluoroSpot Assay

Mouse spleens were harvested at day 7 after the second dose, minced, passed through a cell strainer, and cryopreserved after lysis of red blood cells. Cellular immune responses were measured by IFN-γ FluoroSpot assay according to the manufacturer's instructions (Cat. No. FSP-4246-2 Mabtech, Inc., Cincinnati, OH). Briefly, splenocytes were rested at 37° C., in 5% CO₂ for 3 hours after thawing to allow removal of cell debris. A total of 2.5×105 cells per well in serum-free CTL-Test™ medium (Cellular Technology Limited, Shaker Heights, OH) were added to a 96 well PVDF membrane plate pre-coated with capture monoclonal antibodies and stimulated for 40 hours with peptides, PepTivator ° SARS-CoV-2 Prot_S1 peptide pool consisting of 15-mer peptides with 11 amino acids overlapping, covering the N-terminal S1 domain of the Spike protein of SARS-CoV-2 (Miltenyi Biotec, Auburn, CA) at 0.2 μg/mL and 0.5 μg/mL per peptide, or medium alone. Cells (5×104) were incubated with PMA (0.01 μM)/Ionomycin (0.167 μM) cocktail (BioLegend, San Diego, CA) as a positive control. The tests were set up in duplicates, and the costimulatory anti-CD28 antibody was added to the cells during the incubation. Plates were developed using specific monoclonal detection antibodies and fluorophore-conjugated secondary reagents. Finally, plates were treated with a Fluorescence enhancer (Mabtech) to optimize detection and then air-dried. The spots were enumerated using the CTL ImmunoSpot® S6 Universal Analyzer (Cellular Technology Limited, CTL, Shaker Heights, OH), and the number of antigen specific cytokine-secreting spot forming cells (SFCs) per million cells for each stimulation condition was calculated by subtracting the number of spots detected in the medium only wells.

### V. Flow Cytometry

Cryopreserved splenocytes from day 28 (1-week post-dose 2) were thawed and prepared for flow cytometry analysis. Thawed cells were suspended in 10 mL Roswell Park Memorial Institute Medium (RPMI) supplemented with 10% fetal bovine serum (FBS), centrifuged at 1100× G for 10 minutes and washed twice with phosphate buffered saline (PBS). Mouse FC Receptors were then blocked using TruStainFX (BioLegend) and Zombie red (BioLegend) was used for live/dead determination in all three panels per manufacturers recommendations, followed by two washes with PBS. Cells were then divided for staining (maximum 2.0×106 cells) with three separate panels for broadly phenotyping monocytes, T-cells, and B-cells. The monocyte antibody (directly conjugated) panel consisted of: HLA-Dr (FITC) (eBioscience), F4/80 (BV421), CD1 1 c (BV510), CD1 lb (BV605), Ly-6c (BV711) CCR5 (PE), I-A/I-E (PE-Cy7), IFNγ (APC), CD4 (Ax700), and CD3 (APC-Cy7) (BioLegend); the T-cell panel: TCRb (BV510), CXCR5 (BV605), PD-1 (BV421), FoxP3(FITC), CD3 (PerCP/Cy5.5), LAG-3 (PE/Cy7), ICOS (PE), CD4 (Ax700), Ki67 (Ax647), and CD8 (APC/Cy7) (BioLegend); the B-cell panel: CD27 (BV605), CD3 (BV650), IgG (BV421), (Ki67 (PerCP/Cy5.5), CD38 (PE/Cy7), CD19 (PE), MHCII(I-Ad) (Ax647), CD138 (APC/Cy7), and CD45R (Ax700) (BioLegend). Extracellular staining was conducted using manufacturer recommended concentrations with a 45-minute incubation time at room temperature. Intracellular staining for monocytes was conducted using FACS permeabilizing solution 2 (BD Biosciences) and Cyto-Fast fix/perm buffer set (BioLegend) was used for T-cells and B-cells. Both kits were used per manufacturers' recommendation. After the staining procedures, cells were suspended in PBS containing 1% paraformaldehyde overnight. Samples were acquired using the LSRFortessa Flow Cytometer (BD Biosciences) and analyzed using FlowJo software (BD Biosciences).

### VI. Recombinant Protein Expression and Purification

Plasmids were generated to express the native-like, trimeric, transmembrane (TM)-deleted spike (S) glycoprotein (SdTM) from SARS-CoV-2 strain Wuhan-Hu-1 (Genbank Accession number NC_045512). The SdTM sequence was designed to encode the SARS-CoV-2 S protein sequence spanning Val 16 to Ser1147. The SdTM gene was produced by de novo synthesis in four fragments (Integrated DNA Technologies, Inc, Coralville, IA) and assembled using HIFI DNA Assembly Cloning kit (New England Biolabs Ipswich, MA). The gene was also codon-optimized for expression in Drosophila S2 cells, with an abolished furin cleavage site (RRAR682-685GSAS) between S1 and S2 domains to prevent cleavage and contains a trimerization domain of T4 bacteriophage fibritin (foldon) at the C-terminus. Two additional proline substitutions (KV986-987PP) between the heptad repeat 1 and central helix regions and the removal of the S2′ protease cleavage site (KR814-815GA) were introduced by site-directed mutagenesis to generate the stabilized prefusion structure of S protein (SdTM2P). The proprietary expression vector pUHMS2v containing S gene variants was transfected into Drosophila S2 cells using Expifectamine Sf or Lipofectamine 3000 reagent (Invitrogen, Carlsbad, CA) according to manufacturer's instructions. Stably transformed cell lines were created by selection with culture medium containing hygromycin B at 300 μg/mL. To verify selection, transformants were induced with culture medium containing 200 μM CuSO4. Expression was verified by sodium dodecyl sulfate polyacrylamide gel electrophoresis (SDS-PAGE) and western blotting.

Recombinant S proteins were purified from filtered cell culture supernatants by affinity chromatography (AC) using NETS-activated Sepharose (Cytiva, Marlborough, MA) coupled with 2 mg/ml of column matrix of a his-tagged human angiotensin I converting enzyme 2 (hACE2), which was also produced using the Drosophila S2 cell expression system and purified by Ni-affinity chromatography. Purified recombinant S proteins were concentrated using Amicon filtration devices (EMD Millipore, Billerica, MA), buffer-exchanged into PBS and analyzed by SDS-PAGE and Western blotting. Antigens were quantified by UV absorbance at 280 nm and stored at −80° C.

A conventional immunoaffinity chromatography (IAC) method was also applied to purify SdTM2P proteins. For this, the monoclonal antibody (mAb) CR3022 (provided by Mapp Biopharmaceutical), was coupled to NETS-activated Sepharose at a concentration of 10 mg/mL and used for IAC in tandem with a HiPrep 26/10 desalting column (Cytiva, Marlborough, MA) equilibrated with PBS allowing quick buffer exchange of the eluted protein from low pH buffer into PBS.

VII. Mouse Experiments with Recombinantly Express Spike Protein

Groups (n=7 or 15 per group) of 7 to 10-week-old Swiss Webster mice of both sexes (bred from original breeding stocks obtained from Taconic Biosciences, Germantown, NY) were immunized intramuscularly (i.m.) at days 0 and 21 with 5 μg of SARS-CoV-2 spike proteins SdTM or SdTM2P (purified by hACE2 affinity chromatography) alone or in combination with 1 mg of CoVaccine HT™ adjuvant (Protherics Medicines Development Ltd, a BTG company, London, United Kingdom). The negative control group received equivalent doses of adjuvant only. To determine the optimal antigen and adjuvant dosing, mice were also immunized with either 5, 2.5, or 1.25 μg of SdTM2P (purified by mAb CR3022 IAC) and 1 mg of CoVaccine HT™ or 5 μg of SdTM2P formulated with 0.3 mg CoVaccine HT™. Serum samples were collected on days 7, 14, 28, and 35. Three to five mice from each group were euthanized on the seventh day after the first and second vaccinations and splenectomies were performed for preparation of splenocytes. The remaining four to five mice from each group were euthanized 14 days after the second vaccination and terminal bleeds collected by cardiac puncture.

### VIII. Analysis of Antibodies by Multiplex Microsphere Immunoassay (MIA)

The IgG antibody in mouse sera was measured by a multiplex microsphere-based immunoassay as described previously [9, 29, 30]. Briefly, internally dyed, magnetic MagPlex® microspheres (Luminex Corporation, Austin, TX) were coupled to purified receptor binding domain (RBD-F), spike protein (SdTM2P) or bovine serum albumin (BSA) as control [9, 29]. A mixture of RBD, spike SdTM2P, and BSA-coupled beads (approximately 1,250 beads each) was incubated with diluted sera in black-sided 96-well plates for 3 hours at 37° C. with gentle agitation in the dark. Following two washes with MIA buffer (1% BSA and 0.02% Tween 20 in 1 x PBS), 50 μlof 111g/mL red phycoerythrin (R-PE)-conjugated goat anti-mouse IgG antibodies (Jackson ImmunoResearch, Inc., West Grove, PA) were added and incubated at 37° C. for another 1 hour. After washing twice with the MIA buffer, the beads were resuspended in MAGPIX® drive fluid and analyzed on a MAGPIX® Instrument (Luminex Corporation, Austin, TX).

The median fluorescence intensity (MFI) readouts of the experimental samples were converted to antibody concentrations using purified antibody standards prepared from pooled mouse antiserum to SdTM or SdTM2P as described below. For this, IgG was purified from mouse antisera by protein A affinity chromatography and then subjected to immunoaffinity chromatography (IAC) using SdTM2P-coupled NHS-Sepharose (Cytiva, Marlborough, MA) to select only S-reactive IgG. The concentration of purified S-specific IgG was quantified by measuring the UV absorbance of the solution at 280 nm. The purified anti-spike IgG was diluted to concentrations in the range of 4.88 to 5000 ng/mL and analyzed in the MIA assay. The resulting MFI values were analyzed using a sigmoidal dose-response, variable slope model (GraphPad Prism, San Diego, CA), with antibody concentrations transformed to log10 values. The resulting curves yielded r2 values >0.99 with well-defined top and bottom and the linear range of the curve was determined. The experimental samples were analyzed side-by-side with the antibody standards at different dilutions (1:50, 1:200, 1:1000, 1:40,000, 1:80,000 or 1:160,000) to obtain MFI values that fall within the linear range of the standard curve. The experimental sample IgG concentrations were interpolated from the standard curves using the same computer program. Finally, the interpolated values were multiplied by the dilution factors and plotted as antibody concentrations (ng/mL).

The IgG subclass profile in serum samples was analyzed using IgG subclass-specific secondary antibodies (Southern Biotech, Birmingham, AL), and the ratios of IgG2a/IgG1 and IgG2b/IgG1 were calculated using the MFI readouts at the serum dilution (1:2000) that is within the linear range of the antibody binding standard curve.

IX. Recombinant Vesicular Stomatitis Virus (rVSV) Plaque and Neutralization Assay

Replication-competent rVSV expressing SARS-CoV-2 S protein without cytoplasmic tail (rVSV-SARS-CoV-2-S) was provided by Dr. Andrea Marzi (Laboratory of Virology, National Institute of Allergy and Infectious Diseases) and the virus stocks were amplified in Vero E6 cells. Virus titers were measured on Vero E6 cells in 6-well plates by standard plaque assay. Briefly, 500 μL of serial 10-fold virus dilutions were incubated with 2.5×10⁶ cells/well at 37° C. for 1 hour and then overlaid with DMEM containing 2% fetal bovine serum (FBS) and 1% agarose. Following incubation in a 37° C., 5% CO₂ incubator for 72 hours, cells were fixed and stained with a solution containing 1% formaldehyde, 1% methanol, and 0.05% crystal violet overnight for plaque enumeration.

For the plaque reduction neutralization test (PRNT) using rVSV- SARS-CoV-2-S, pooled or individual mouse serum samples were heat-inactivated at 56° C. for 30 minutes. Eight 3-fold serial dilutions of serum samples starting at a final 1:10 dilution were prepared and incubated with 100 plaque-forming units (PFU) of rVSV-SARS-CoV-2-S at 37° C. for 1 hour. Antibody-virus complexes were added to Vero E6 cell monolayers in 6-well plates and incubated at 37° C. for another hour followed by addition of overlay media. Three days later, the plaque visualization and enumeration steps were carried out as described in the plaque assay. The neutralization titers (PRNT₅₀) were defined as the highest serum dilution that resulted in 50% reduction in the number of plaques.

### X. Wild-Type SARS-CoV-2 Virus Neutralization Assay

PRNT was also performed in a biosafety level 3 facility at BIOQUAL, Inc. (Rockville, MD) using 24-well plates. Mouse sera pooled from individual mice within each group, were diluted to 1:10, and a 1:3 serial dilution series was performed 11 times. Diluted samples were then incubated with 30 plaque-forming units of wild-type SARS-CoV-2 (USA-WA1/2020, BEI Resources NR-52281) in an equal volume of culture medium (DMEM with 10% FBS and gentamicin) for 1 hour at 37° C. The serum-virus mixtures were added to a monolayer of confluent Vero E6 cells and incubated for 1 hour at 37° C. in 5% CO₂. Each well was then overlaid with 1 ml of culture medium containing 0.5% methylcellulose and incubated for 3 days at 37° C. in 5% CO₂. The plates were then fixed with methanol at −20° C. for 30 minutes and stained with 0.2% crystal violet for 30 min at room temperature. Neutralization titers (PRNT₅₀) were defined as the highest final serum dilution that resulted in 50% reduction in the number of plaques.

### XI. Splenocyte Preparation and FluoroSpot

Three or five mouse spleens from each group were harvested seven days after the first and second vaccinations, and single cell suspensions were prepared using a gentle MACS Dissociator (Miltenyi Biotec, Auburn, CA). The cells were passed through a cell strainer, resuspended in freezing medium containing 90% FBS and 10% dimethyl sulfoxide (DMSO) after lysis of red blood cells, and cryopreserved in liquid nitrogen. FluoroSpot assay was performed using mouse IFN-γ FluoroSpotPLUS kit according to the manufacturer's instructions (Mabtech, Inc., Cincinnati, OH). Briefly, splenocytes were rested in a 37° C., 5% CO₂ incubator for 3 hours after rapidly thawing in a 37° C. water bath followed by slow dilution with culture medium to allow removal of cell debris. A total of 2.5×10⁵ cells per well in RPMI-1640 medium supplemented with 10% FBS, penicillin (100 units/mL) and streptomycin (100 μg/mL) were added in a 96 well PVDF membrane plate pre-coated with capture monoclonal antibodies. The cells were stimulated for 24 hours with a peptide pool consisting 17-mer peptides with 10 amino acids overlapping, covering the spike protein of SARS-CoV-2 (BEI Resources NR-52402) at 10 μg/mL or medium containing equal concentration of DMSO (0.05%) as negative control. Fifty thousand cells were incubated with cell activation cocktail (BioLegend, San Diego, CA) at 1:500 dilution containing phorbol-12-myristate 13-acetate (PMA, 0.081 μM) and Ionomycin (1.3386 μM) as positive control. Each stimulation condition was set up in triplicate. Plates were developed using specific monoclonal detection antibodies and fluorophore-conjugated secondary reagents. Finally, plates were treated with a Fluorescence enhancer (Mabtech) to optimize detection and then air-dried. The spots were enumerated using the CTL ImmunoSpot® S6 Universal Analyzer (Cellular Technology Limited, CTL, Shaker Heights, OH), and the number of antigen-specific cytokine secreting spot forming cells (SFCs) per million cells for each stimulation condition was calculated by subtracting the number of spots detected in the medium only wells.

### XII. Statistical Analysis

Statistical analysis was performed using one sample t test or Mann-Whitney t test to compare the neutralization titers, IgG subclass antibody profile, and IFN-γ secreting response between adjuvanted and unadjuvanted groups. The difference in the neutralizing antibody titers and the numbers of IFN-γ secreting cells between groups receiving different dosages of vaccines was determined by one-way ANOVA with Tukey's multiple comparison test. The correlation between the measured neutralization titers using rVSV-SARS-CoV-2 and authentic SARS-CoV-2 was analyzed by Pearson correlation analysis.

### XIII. Non-Human Primate Experiments

Non-naïve cynomolgus macaques (N=3/group) were administered vaccines with protein SdTM2P and CoVaccine HT™ either as a liquid stock or lyophilized at 10mg/ml in the presence of 9.5% trehalose. Specific formulations included 5 μg SdTM2P+5 mg lyophilized CoVaccine HT™, 25 μg SdTM2P +5 mg liquid CoVaccine HT™, 25 μg SdTM2P+5 mg lyophilized CoVaccine HT™ and 10 mg lyophilized CoVaccine HT™ (“CoV”) alone. Neutralizing antibodies (wild-type virus) were assessed as described above, and reported as either the plaque reduction neutralization titer at 50% (PRNT50) or 90% (PRNT90).

### Results

A. Murine Immunization with SARS-CoV-2 Spike S1 Proteins

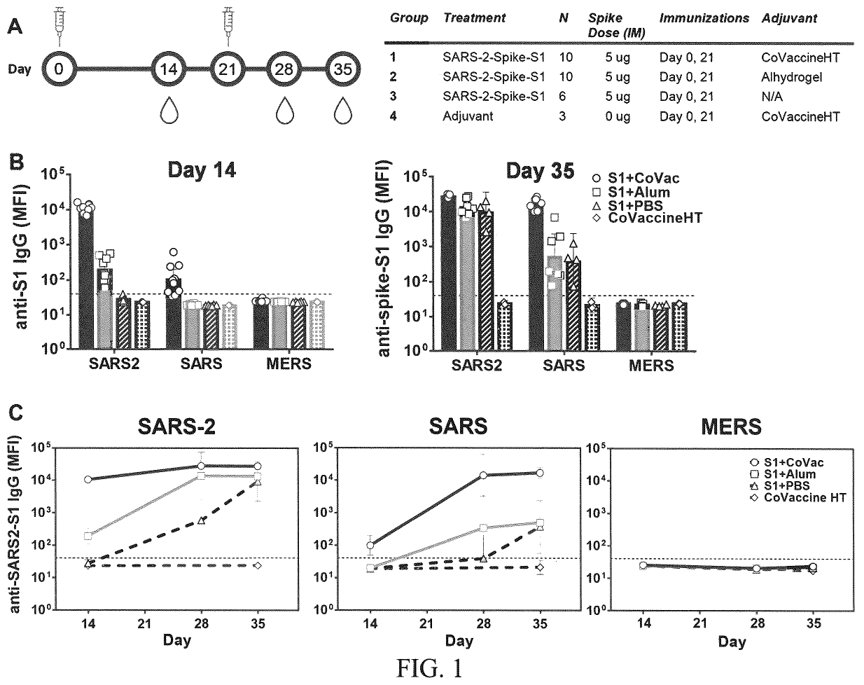

Neutralizing antibodies of SARS-CoV-2 largely target the receptor binding domain present within the Spike protein subdomain 1 (S1) (Jiang, S., Hillyer, C. & Du, L. Neutralizing Antibodies against SARS-CoV-2 and Other Human Coronaviruses: (Trends in Immunology 41, 355-359; 2020). Trends Immunol 41, 545, doi :10.1016/j .it.2020.04.008 (2020)). Therefore, BALB/c mice were given two doses of commercially available Spike-S1 (Sino Biologic 40591-V05H1), intramuscularly (I.M.), 21 days apart (FIG. 1A). To test whether adjuvants may alter immunological responses to the immunogen, mice were divided into four groups based on vaccine formulation. The S1+CoVac, S1+Alum, and S1+PBS groups received SARS-CoV-2 spike S1 mixed with either CoVaccine HT™ (“CoVac”), Alhydrogel® (“Alum”), or PBS, respectively. One group received CoVaccine HT™ alone as an adjuvant control (FIG. 1A).

### B. Adjuvants Alter Immunogenicity and Specificity to Immunization

Serum analysis revealed high reactivity of SARS-CoV-2 S1 specific IgG antibodies in S1+CoVac after a single dose while S1+Alum titres were near baseline (FIG. 1B). Only one animal showed a detectable titre in the antigen alone group at this time point. The only cross reactivity observed after the first dose was to SARS-CoV S1. On day 35, S1+Alum and S1+PBS displayed significantly higher antibody responses compared to day 14 and variations among individual animals were reduced. S1+CoVac treated animals on day 35 consistently showed very high antibody responses in every animal. Similarly, cross-reactivity with SARS-CoV S1 was greatly increased for all groups on day 35 (FIG. 1B). As expected, due to its much lower sequence homology and the use of a different cellular receptor (DPP4) for viral entry the SARS-CoV-2 S1 did not induce IgG responses to MERS-CoV Si.

In human cases, high titres of SARS-CoV-2 spike-specific IgG are associated with subclinical infections while low titres are associated with increased severity in disease (Sun, B. et al. Kinetics of SARS-CoV-2 specific IgM and IgG responses in COVID-19 patients. Emerg Microbes Infect 9, 940-948, doi :10.1080/22221751.2020.1762515 (2020)). Therefore, antibody response kinetics may be an important factor for a vaccine candidate. Time-course analysis of IgG responses reveal that adjuvanted S1 may be crucial for strong, early IgG responses with SARS-CoV-2 specificity while a second dose may solidify the response and increase cross-reactivity (FIG. 1C).

### C. CoVaccine HT™ Improves IgG Titres to SARS-CoV-2 and SARS-CoV S1 Proteins

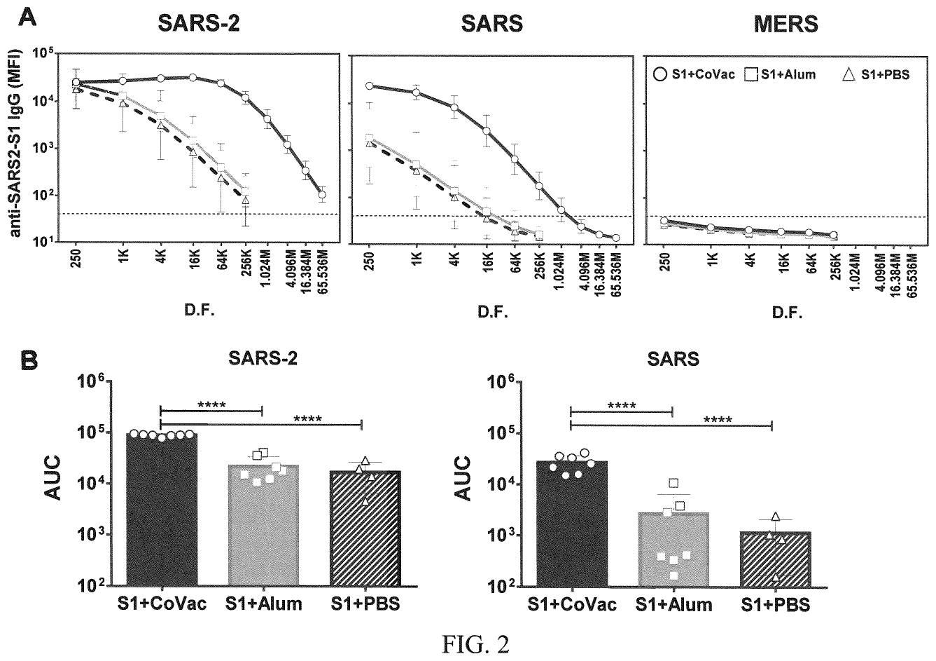

To further interrogate the matured IgG responses, sera from day 35 were titrated in a four-fold dilution series starting at 1/250 and analyzed by microsphere immunoassay (MIA). The S1+Alum and S1+PBS groups showed reactivity to SARS-CoV-2 S1 when diluted up to 1/256,000, indicating an abundance of antigen-specific IgG in the sera (FIG. 2A). Titrating sera from S1+CoVac however, revealed saturating levels of IgG for five dilutions and detectable IgG levels were present down to a 1/65.5 million dilution. Antiserum to S1+CoVac also showed significantly greater cross reactivity to SARS-CoV S1 compared to the other groups. All groups remained negative for cross reactivity to MERS-CoV S1 (FIG. 2A). These data suggest that immunization with SARS-CoV-2 S1 and CoVaccine HT™ elicits robust antigen-specific IgG response with the expected cross-reactivity profile to include SARS-CoV S1.

D. Increased IgG Subclass Diversity and Enhanced Viral Neutralizing Antibody Titers with CoVaccine HT™.

Adjuvants serving as TLR4 agonists, such as postulated for CoVaccine HT™, elicit a primarily Th1 type response (Matsuoka, Y. et al. Requirement of TLR4 signaling for the induction of a Th1 immune response elicited by oligomannose-coated liposomes. Immunol Len 178, 61-67, doi:10.1016/j.imlet.2016.07.016 (2016); Perrin-Cocon, L. et al. Th1 disabled function in response to TLR4 stimulation of monocyte-derived DC from patients chronically-infected by hepatitis C virus. PLoS One 3, e2260, doi:10.1371/journal.pone.0002260 (2008)). Meanwhile, Alhydrogel® facilitates a mainly Th2 type response, possibly through NOD-like receptor signaling (Li, H., Willingham, S. B., Ting, J. P. & Re, F. Cutting edge: inflammasome activation by alum and alum's adjuvant effect are mediated by NLRP3. J Immunol 181, 17-21, doi:10.4049/jimmuno1.181.1.17 (2008); Morefield, G. L. et al. Role of aluminum-containing adjuvants in antigen internalization by dendritic cells in vitro. Vaccine 23, 1588-1595, doi:10.1016/j.vaccine.2004.07.050 (2005)).

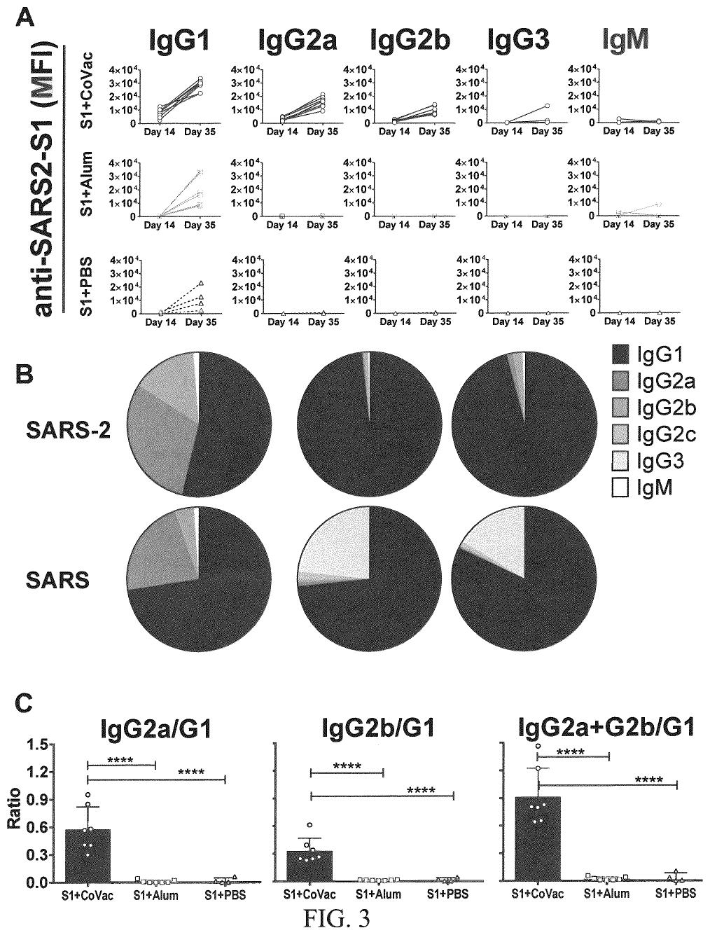

IgG subclass analysis can be used to determine if a Th1 or Th2 response may have been more prominent. Sera from each S1+adjuvant group were analyzed for their subclass composition (FIG. 3). Consistent with previous findings, the S1+CoVac group displayed a diverse immunoglobulin response composed of IgG1, IgG2a, and IgG2b subclasses all of which were further elevated after a second dose of vaccine. Low levels of IgG3 were also observed.

Alternatively, the Alum and antigen alone groups primarily produced an IgG1 response with some detectable IgM in the Alum group, representing a classical Th2-biased humoral response. Heterogeneous subclass populations such as those observed in the S1+CoVac group are typically associated with Th1 responses while IgG1 is characteristic of a Th2 response. To further investigate the nature of these adjuvant effects, the subclass data were stratified to analyze ratios of Th1 vs Th2 subclasses (FIG. 3C). This analysis shows clearly that of the three tested formulations, only S1+CoVac induced a relatively balanced humoral response. Furthermore, only the S1+CoVac formulation was able to induce detectable SARS-CoV-2 neutralizing antibody titers as demonstrated in a plaque reduction neutralization test using wildtype virus (Table 1). PRNT90 and PRNT50 titres for this formulation indicate potent neutralization (1:1620).

TABLE 1
SARS-CoV-2 neutralization titres

| Group ID | Titre (PRNT₉₀) | Titre (PRNT₅₀) |
|---|---|---|
| S1 + CoVac | 1620 | 1620 |
| S1 + Alum | <20 | <20 |
| S1 + PBS | <20 | <20 |
| CoVaccine^{HT} | <20 | <20 |

### E. Adjuvant Effect on the SARS-CoV-2 Sl-Specific Cytokine Responses

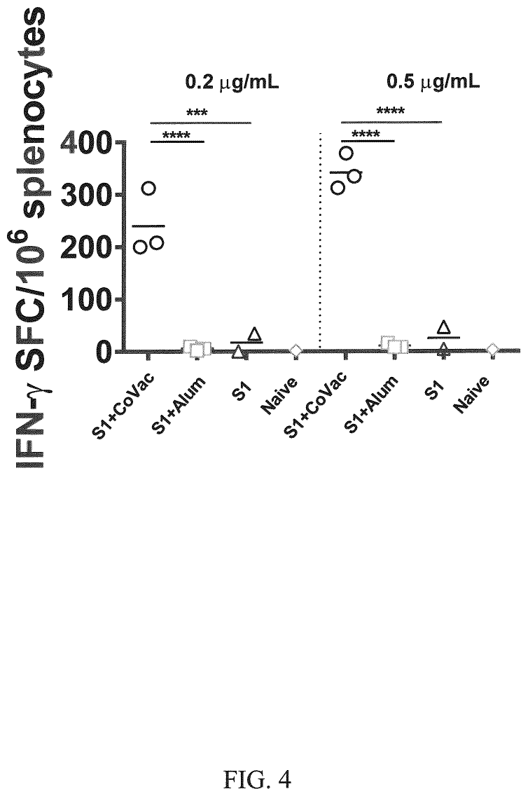

We assessed the adjuvant effect of CoVaccine HT™ and Alum on the cellular immune responses directed against SARS-CoV-2 S1 using an IFN-γ FluoroSpot assay. Individual mouse spleens from each group harvested at day 7 post second immunization were processed and single cell suspensions stimulated with SARS-CoV-2 S1 peptides. The number of IFN-γ secreting cells from the mice given CoVaccine HT™ was significantly higher than those for mice given Alum or S1 antigen only at two different peptide concentrations (Fig.4). Splenocytes from unvaccinated (naïve) mice did not respond to S1 peptide stimulation.

The COVID-19 pandemic has spurred global efforts to rapidly develop vaccines against SARS-CoV-2. Many vaccine strategies are being explored, however, only non-replicating viral vectors, recombinant protein, DNA, and RNA platforms have reached phase-I clinical trials 6. The number of clinically applied adjuvants are limited and include alum and newer formulations such as MF59 and AS03, both oil-in-water emulsions using squalene (Prevention, C. f. D. C. a. What is an adjuvant and why is it added to a vaccine? https://www.cdc.gov/vaccinesafety/concerns/adjuvants.html Oct. 24, 2018)). The small number of adjuvants approved for clinical use has limited vaccine development in the past and impacts current clinical trials of SARS-CoV-2 vaccines. Many approaches use no adjuvant, Alum, MF59, or AS03, however, Novavax is testing the experimental adjuvant Matrix-MTM (Gupta, T. & Gupta, S. K. Potential adjuvants for the development of a SARS-CoV-2 vaccine based on experimental results from similar coronaviruses. Int Immunopharmacol 86, 106717, doi:10.1016/j.intimp.2020.106717 (2020)). While Alum is known to primarily enhance a Th2 response, Matrix-W™ and CoVaccine HT™ have both shown to elicit a Th1 response with recombinant subunits. Due to the previously observed potential for enhanced immunopathology associated with primarily Th2-targeted anti-SARS-CoV or anti-MERS-CoV vaccines, the development of a COVID-19 vaccine may require testing of a multitude of adjuvants to elicit protective immune responses to SARS-CoV-2. The squalane-in-water based adjuvant, CoVaccine HT™ , has previously shown to induce potent virus neutralization antibody titres and protective efficacy in mice and non-human primates to several infectious agents (Medina, L. O. et al. A Recombinant Subunit Based Zika Virus Vaccine Is Efficacious in Non-human Primates. Front Immunol 9, 2464, doi:10.3389/fimmu.2018.02464 (2018); Lehrer, A. T. et al. Recombinant proteins of Zaire ebolavirus induce potent humoral and cellular immune responses and protect against live virus infection in mice. Vaccine 36, 3090-3100, doi:10.1016/j.vaccine.2017.01.068 (2018); Mandi Abdel Hamid, M. et al. Vaccination with Plasmodium knowlesi AMA1 formulated in the novel adjuvant co-vaccine HT protects against blood-stage challenge in rhesus macaques. PLoS One 6, e20547, doi:10.1371/journal.pone.0020547 (2011); Kusi, K. A. et al. Safety and immunogenicity of multi-antigen AMA1-based vaccines formulated with CoVaccine HT and Montanide ISA™ 51 in rhesus macaques. Malar J 10, 182, doi:10.1186/1475-2875-10-182 (2011)).

In this study, we investigated the immunogenicity of recombinant SARS-CoV-2 Spike-S1 alone or in combination with Alum or CoVaccine HT™ as potential adjuvants. Overall, we observed the most potent humoral and cellular immune responses, including neutralizing antibody responses in the CoVaccine HT™ study group. Day 14 titres (post-dose 1) indicate that this formulation may even be efficacious after administration of a single dose, however, this was not investigated in the current study. Neither antigen alone nor the combination with Alum was able to induce detectable neutralizing antibodies with the model antigen utilized in this study. This may be due to slower response kinetics caused by antigen presentation, subclass homogeneity, or Th2 restricted immune responses compared to administering S1 with CoVaccine HT™.

Immunogenicity of protein subunit vaccines is often inferior in generating robust immune responses compared to other platforms such as those based on live attenuated viruses. As seen here, the (monomeric) S1 domain alone is not adequate for generating a high titre immune response. The addition of CoVaccine HT™ improved antibody titres and response kinetics and proved to induce high titres of antibodies neutralizing wild-type SARS-CoV-2. It has been shown by others that SARS-CoV-2 S1 IgG titres correlate with viral neutralization in humans (Robbiani, D. F. et al. Convergent antibody responses to SARS-CoV-2 in convalescent individuals. Nature, doi:10.1038/s41586-020-2456-9 (2020)). Virus neutralizing responses in rabbits after two immunizations with 50 μg of SARS-CoV-2 S1 and EMULSIGEN® adjuvant were comparatively low at 1:160 in the wild-type neutralization assay, further demonstrating the importance of an adjuvant with desirable properties (Ravichandran, S. et al. Antibody signature induced by SARS-CoV-2 spike protein immunogens in rabbits. Sci Transl Med 12, doi:10.1126/scitranslmed.abc3539 (2020)). In our study, post-dose 1 titres in the S1+CoVac group resemble post dose 2 titres with Alum or no adjuvant and may suggest at least partial protection after a single dose. Generating potent immunity after a single dose is an attractive target for any SARS-CoV-2 vaccine in development and may improve the impact of a vaccine on the further course of the pandemic.

The high potency for SARS-CoV-2 S1 in the CoVaccine HT™ formulation may be attributable to the observed immunoglobulin subclass diversity. This indicates CoVaccine HT™ may efficiently induce class switching often considered to increase antibody affinity. Furthermore, a broad IgG subclass composition is key for inducing complement-mediated antibody effector functions as well as neutralization and opsonization, which are typically essential for mitigating viral infections. The ideal antibody population has yet to be elucidated for combating SARS-CoV-2. However, our murine serological data suggests kinetics and subclass diversity may be key to developing effective immune responses. Additionally, we have demonstrated that CoVaccine HT™ is not only a suitable adjuvant for vaccination but is preferable to Alhydrogel® given the quality of the humoral response due to rapid onset, balance, overall magnitude of the response, as well as significantly greater cell-mediated immune responses.

Concerns have been raised regarding antibody dependent enhancement (ADE) with SARS-CoV-2 infection or immunization. This phenomenon occurs when non-neutralizing or poorly binding antibodies interact with Fc receptors on antigen presenting cells and facilitate infection. This interaction increases pro-inflammatory cytokine production which exacerbates immunopathology (Iwasaki, A. & Yang, Y. The potential danger of suboptimal antibody responses in COVID-19. Nat Rev Immunol 20, 339-341, doi:10.1038/s41577-020-0321-6 (2020)). ADE was previously observed with SARS-CoV infection by anti-spike antibodies through the FcγR and FcγRII pathways (Jaume, M. et al. Anti-severe acute respiratory syndrome coronavirus spike antibodies trigger infection of human immune cells via a pH- and cysteine protease-independent FcgammaR pathway. J Virol 85, 10582-10597, doi:10.1128/JVI.00671-11 (2011); Yip, M. S. et al. Antibody-dependent enhancement of SARS coronavirus infection and its role in the pathogenesis of SARS. Hong Kong Med J 22, 25-31 (2016)).

In respiratory syncytial virus infections, a Th2 response alone can lead to aberrant immune responses associated with ADE caused by either a subsequent infection or prior immunization (Graham, B. S. et al. Priming immunization determines T helper cytokine mRNA expression patterns in lungs of mice challenged with respiratory syncytial virus. J Immunol 151, 2032-2040 (1993); Kim, H. W. et al. Respiratory syncytial virus disease in infants despite prior administration of antigenic inactivated vaccine. Am J Epidemiol 89, 422-434, doi:10.1093/oxfordjournals.aje.a120955 (1969)). For these reasons, caution may be warranted when using Alum as an adjuvant for SARS-CoV-2, at least for the subunit protein used in this study. Additionally, our data suggest that antigen alone may also drive a predominantly Th2 type response with recombinant S1 antigen immunization in mice. In contrast, the subunit protein with CoVaccine HT™ produces neutralizing antibodies while boosting Th1 responses which may increase durability, vaccine safety, and efficacy.

Other oil in water emulsion adjuvants such as MF59 and AS03 utilize squalene, a shark fat derived product (Blom, A. G. & Hilgers, L. A. Sucrose fatty acid sulphate esters as novel vaccine adjuvants: effect of the chemical composition. Vaccine 23, 743-754, doi:10.1016/j.vaccine.2004.07.021 (2004); Allison, A. C. Squalene and squalane emulsions as adjuvants. Methods 19, 87-93, doi:10.1006/meth.1999.0832 (1999); Garcon, N., Vaughn, D. W. & Didierlaurent, A. M. Development and evaluation of AS03, an Adjuvant System containing alpha-tocopherol and squalene in an oil-in-water emulsion. Expert Rev Vaccines 11, 349-366, doi:10.1586/erv.11.192 (2012)). Squalane may be advantageous as this is a plant derived product, which may be favorable both ideologically and immunologically to a population. This offers the opportunity to produce a vaccine formulation absent of animal components, increasing its safety profile.

F. Drosophila S2 Cell-Expressed Recombinant S Proteins Adjuvanted with CoVaccine HT™ Induce Robust IgG Antibody Responses.

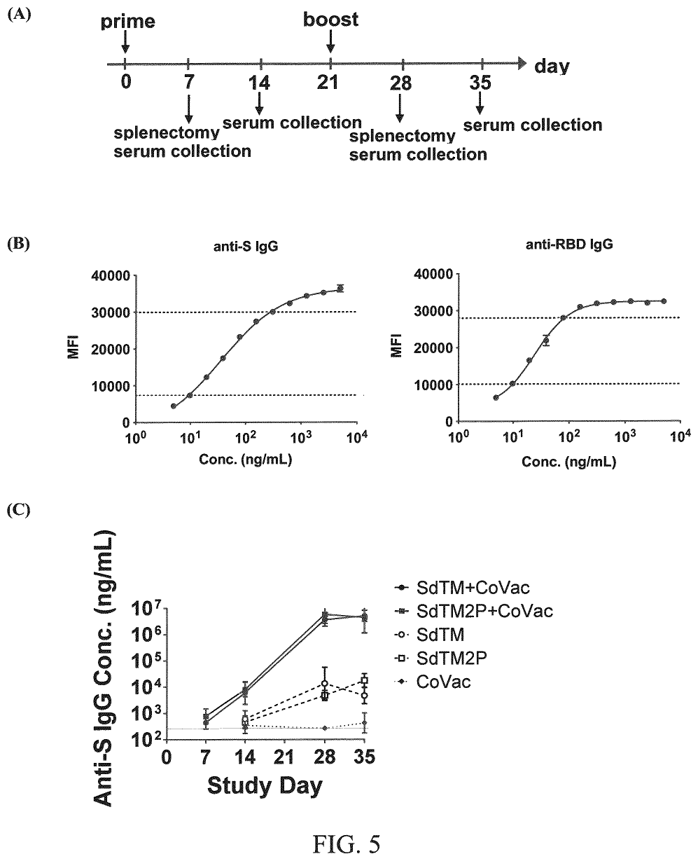

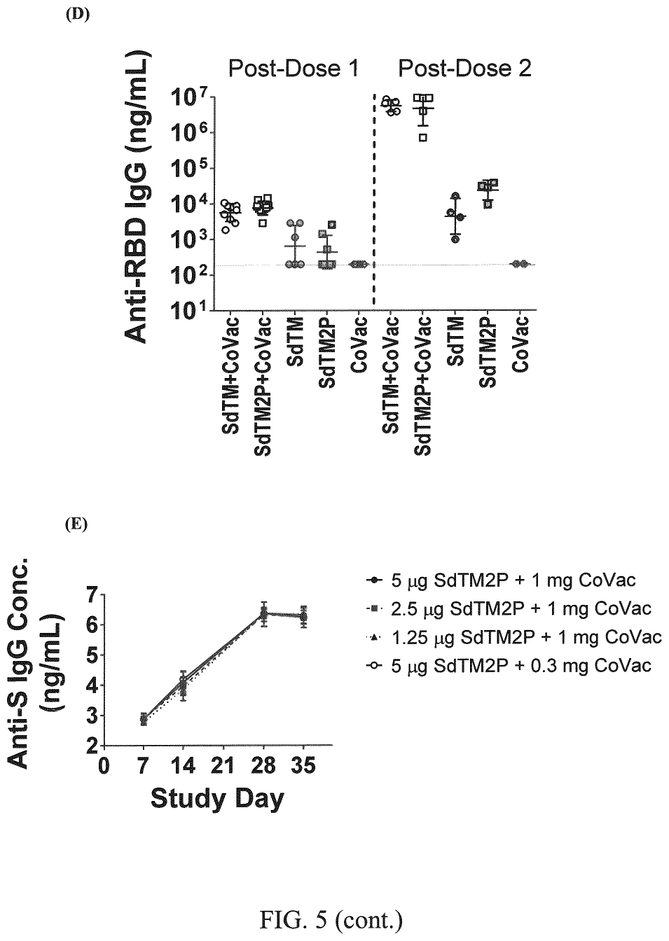

Using the Drosophila S2 cell expression system, we generated trimeric SARS-CoV-2 S protein devoid of transmembrane domain (SdTM) as well as a stabilized prefusion structure of S protein (SdTM2P). To explore different methods for affinity purification of S protein, we first employed a novel method in which we utilized human angiotensin I converting enzyme 2 (hACE2) protein, a known receptor for SARS-CoV-2, for AC purification of S proteins. The immunogenicity of recombinant S proteins was evaluated with and without CoVaccine HT™ adjuvant in outbred Swiss Webster mice. Animals were immunized with one or two doses at a 3-week interval by intramuscular injection with 5 μg of SARS-CoV-2 SdTM or SdTM2P (purified by hACE2 AC) alone or formulated with 1 mg CoVaccine HT™ (FIG. 5A). We developed a quantitative IgG assay in which purified anti-S polyclonal antibody was used as a standard and therefore the antigen-specific IgG levels are expressed as concentration (ng/mL) (FIG. 5B). Analysis of IgG antibody in sera obtained on days 7, 14, 28, and 35 indicated that the co-formulation of CoVaccine HT™ with SdTM or SdTM2P significantly increased anti-S IgG antibody levels over those induced by proteins alone (FIG. 5C). In addition, two doses of CoVaccine HT™ adjuvanted SdTM or SdTM2P elicited high levels of IgG antibodies to RBD, which is the target of neutralizing antibodies (FIG. 5D).

To further optimize vaccine formulations, we evaluated the IgG antibody responses in mice that received a lower dosage (2.5 or 1.25 μg) of SdTM2P proteins or that received a reduced amount (0.3 mg) of adjuvant. The results demonstrated that vaccination with reduced amounts of antigen or adjuvant did not decrease the levels of antigen-specific IgG antibodies (FIG. 5E).

G. Recombinant S Proteins Adjuvanted with CoVaccine HT™ Elicit Potent Neutralizing Antibody Responses.

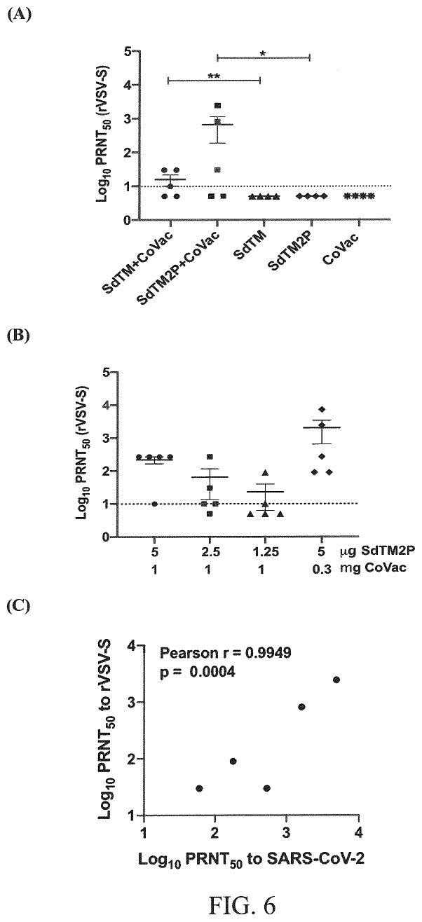

We next examined whether our vaccine candidates induced neutralizing antibody responses. Measurement of SARS-CoV-2 neutralizing antibodies requires biosafety level 3 (BSL3) laboratory facilities, and therefore, alternative assays have been developed, such as one utilizing replication-competent rVSV-ΔG expressing SARS-CoV-2 S glycoprotein (rVSV-SARS-CoV-2-S) [31, 32]. We first compared the neutralizing antibody titers of pooled mouse sera using both authentic SARS-CoV-2 and rVSV-SARS-CoV-2-S (Table 2) and found that the titers are significantly correlated between these two assays (FIG. 6C).

TABLE 2
Virus neutralizing antibody titers against SARS-CoV-2 and rVSV-
SARS-CoV-2-S in pooled antisera from mice immunized with two
doses of S proteins formulated with CoVaccine HT ™ adjuvant

| | | PRNT₅₀^{b} | PRNT₅₀^{b} |
|---|---|---|---|
| Immunogen^{a} | Adjuvant^{a} | (SARS-CoV-2) | (rVSV-SARS-CoV-2-S) |
| 5 μg SdTM | 1 mg CoVaccine HT ™ | 540 | 30 |
| 5 μg SdTM2P | 1 mg CoVaccine HT ™ | 1620 | 810 |
| 5 μg SdTM | None | ND^{c} | ND |
| 5 μg SdTM2P | None | ND | ND |
| None | 1 mg CoVaccine HT ™ | ND | ND |
| 5 μg SdTM2P | 1 mg CoVaccine HT ™ | NA^{d} | 270 |
| 2.5 μg SdTM2P | 1 mg CoVaccine HT ™ | 180 | 90 |
| 1.25 μg SdTM2P | 1 mg CoVaccine HT ™ | 60 | 30 |
| 5 μg SdTM2P | 0.3 mg CoVaccine HT ™ | 4860 | 2430 |

^{a}Two vaccinations with protein with or without adjuvant given intramuscularly
^{b}Pooled antiserum (n = 3 or 5 per group) collected 14 days post-dose 2. Titer equals the highest serum dilution yielding 50% reduction in plaque counts.
^{c}ND: not detected
^{d}NA: Assay results not available

To further evaluate the neutralizing antibodies of individual animals, we employed only the PRNT using rVSV-SARS-CoV-2-S. The PRNT₅₀ titers of sera obtained from mice vaccinated with SdTM or SdTM2P in combination with CoVaccine HT™ were significantly higher than those from animals receiving protein alone (FIG. 6A), suggesting that the CoVaccine HT™ adjuvant consistently enhances induction of neutralizing antibody responses. Furthermore, a trend of dose-dependent decreases of neutralization titers was observed when animals were given lower dosages of SdTM2P proteins (FIG. 6B). Of note, the serum PRNT50 titers of mice receiving a lower amount (0.3 mg) of CoVaccine HT™ were comparable or even higher than those of mice given 1 mg of adjuvant indicating that the optimal vaccine formulation may be achieved using lower dosages of adjuvant. The results indicate that as little as 2.5 μg of S protein antigen with 0.3 mg of adjuvant might be sufficient to induce potent neutralizing antibody responses.

H. Recombinant S Proteins in combinAtion with CoVaccine HT™ Induce a Balanced IgG Subtype Antibody Response.

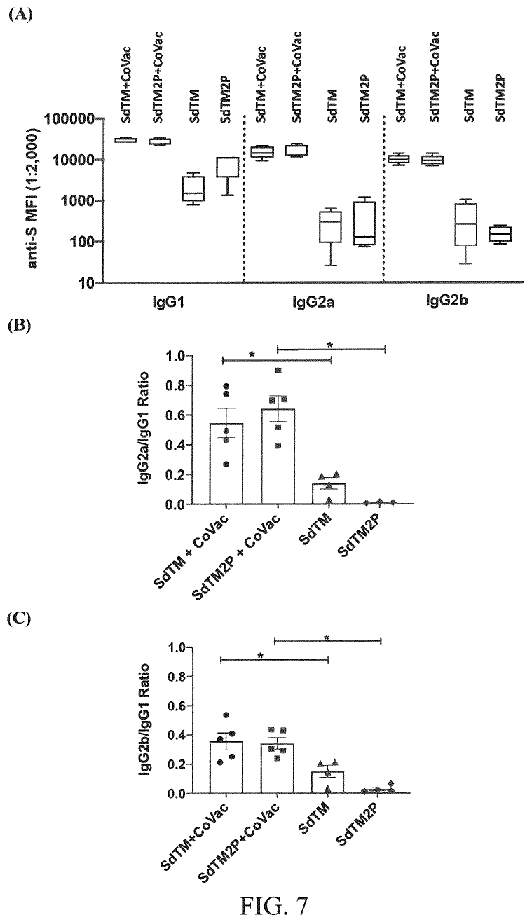

Vaccine-associated enhanced respiratory disease (VARED) has been reported in infants and young children immunized with inactivated whole virus vaccine against respiratory syncytial virus (RSV) and measles virus [33-35], and associated with Th2-biased immune responses [36]. A similar pulmonary immunopathology was also observed in animals immunized with SARS-CoV vaccines [37-39]. Thus, we evaluated the balance of Th1 and Th2 by comparing the levels of S-specific IgG2a/b and IgG1, which are indicative of Th1 and Th2 responses, respectively. Both SdTM and SdTM2P adjuvanted with CoVaccine HT™ elicited high anti-S IgG2a/2b and IgG1 subclass antibodies whereas protein alone induced high IgG1 with lower IgG2a and IgG2b (FIG. 7A). To further assess the effect of CoVaccine HT™ adjuvanticity on IgG subclass antibody profiles, the ratios of IgG2a versus IgG1 as well as IgG2b versus IgG1 were calculated. The results indicate that CoVaccine HT™ enhanced the induction of Th1 responses as evidenced by the significantly higher ratios of IgG2a/IgG2b versus IgG1 obtained from groups given either SdTM or SdTM2P with adjuvant compared to those without adjuvant (FIGS. 7B, 7C).

### I. CoVaccine HT™ Adjuvanted SARS-CoV-2 Recombinant S Protein Vaccine Elicits IFN-γ T Cell Response.

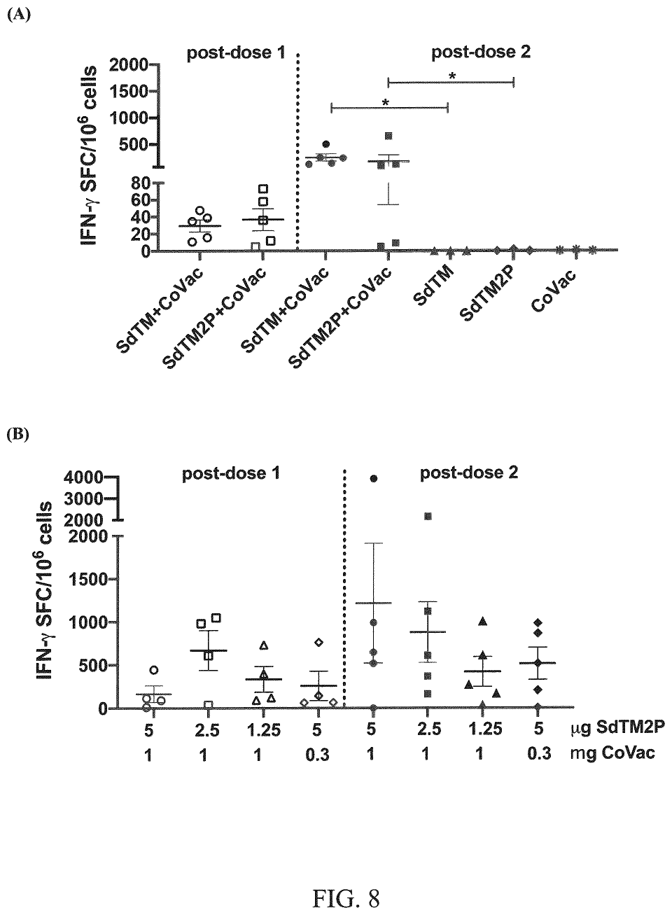

To assess vaccine-induced T cell responses, we analyzed the number of IFN-γ secreting cells after ex vivo stimulation of splenocytes with SARS-CoV-2 spike peptides by a FluoroSpot assay. Stimulation of cells prepared from mice receiving 2 doses of SdTM or SdTM2P with CoVaccine HT™ resulted in robust production of IFN-γ secreting cells (FIG. 8A). Interestingly, one dose of CoVaccine HT™ -adjuvanted S proteins also elicited a rapid IFN-γ secreting response (FIG. 8A). Additionally, a slight decrease in the numbers of IFN-γ secreting cells was observed when mice received a lower amount (2.5 or 1.25 μg) of adjuvanted SdTM2P proteins or a lower amount (0.3 mg) of CoVaccine HT™ (FIG. 8B). However, these differences were not statistically significant. Altogether, our platform shows great potential towards achieving a rapid, robust, and Th1 -focused T cell response even in outbred populations.

### J. CoVaccine HT™ Adjuvanted SARS-CoV-2 Recombinant S PROTEIN VACCINE STRONG NEUTRALIZING Antibody Responses in Non-Human Primates.

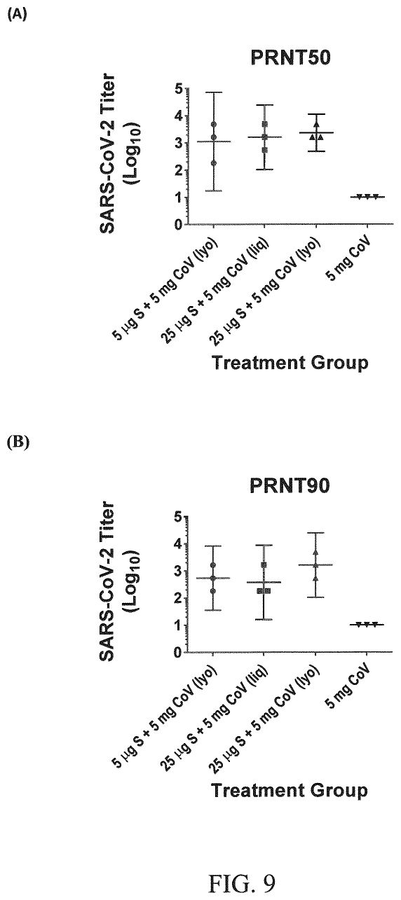

As shown in FIG. 9, a robust antibody response was generated with 5 mg of CoVaccine HT™ used in either the liquid or lyophilized formulation.

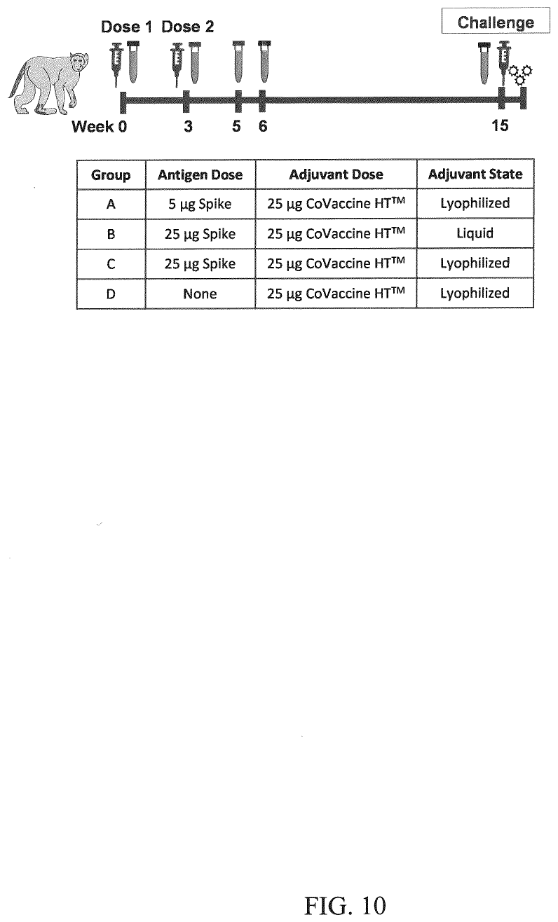

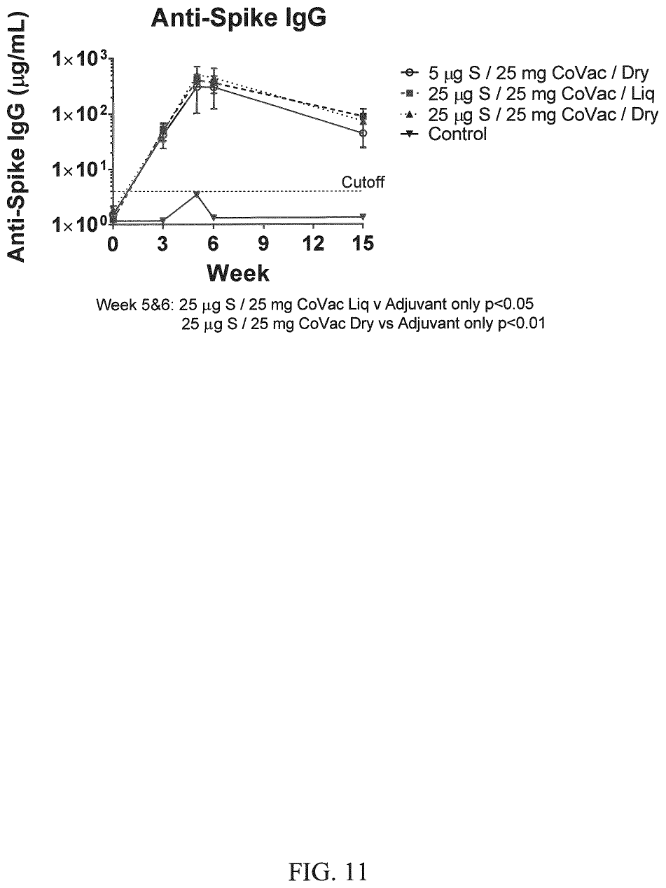

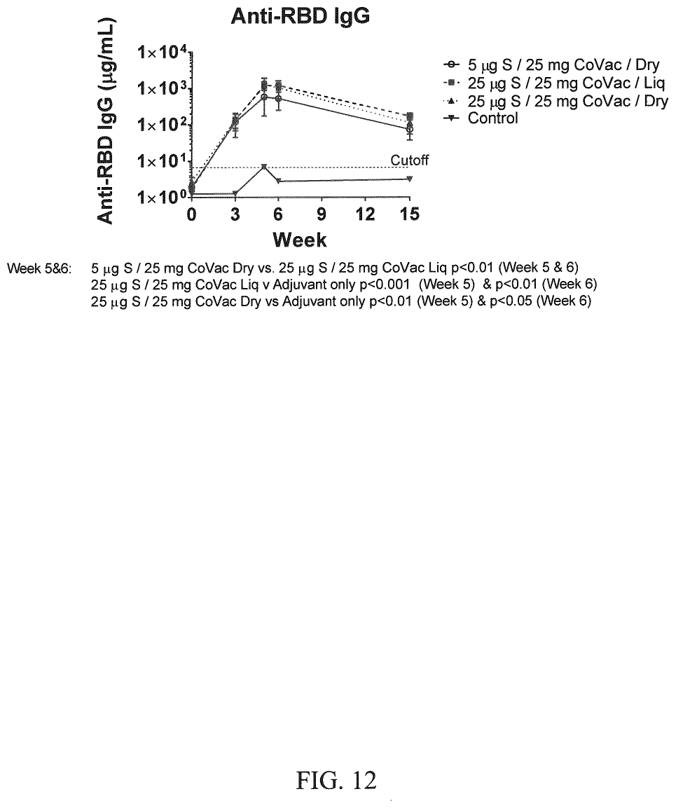

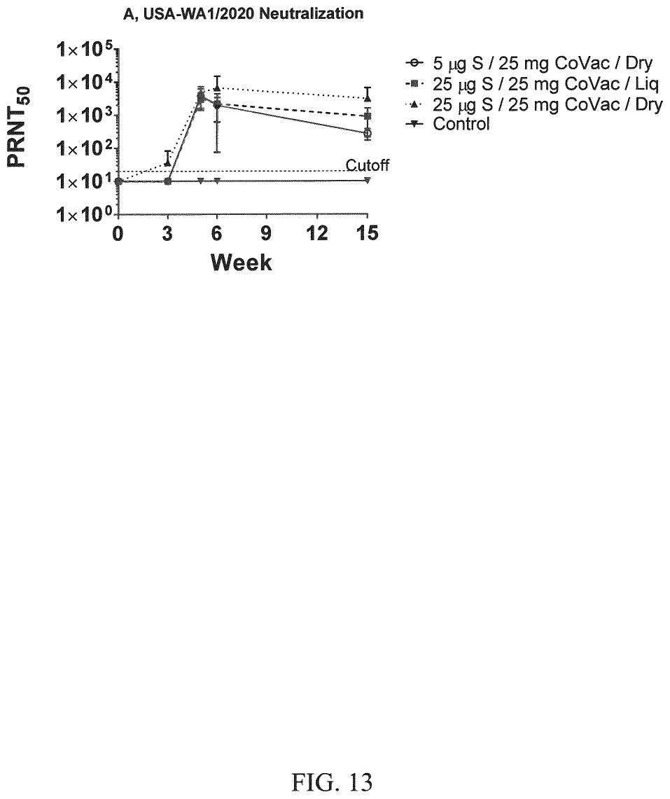

Immunogenicity testing in non-human primates (cynomolgus macaques) was undertaken with a dose level informed based on non-human primate studies with other viral glycoprotein-based candidate vaccines. Two formulations were tested containing 25 μg of antigen SdTM2P in non-human primates (10-fold more than the 2.5 μg shown to be efficacious in mice) and 5 mg of CoVaccine HT™ (10-fold more than the 0.3-1.0 mg range shown to be efficacious in mice) (FIG. 10). CoVaccine HT™ was applied in both liquid and lyophilized formats to demonstrate robustness of the responses to different product storage formats. No difference in immunogenicity was observed with the different CoVaccine HT™ formats (FIGS. 11-12). Moreover, comparison to a group with a lower antigen dose level (5 μg) demonstrated a robust antibody response was achieved with the adjuvant, even as antigen concentrations were decreased (antigen sparing). Overall IgG titers were also determined and found to be potent, with significant levels of IgG seen as early as 14 days after the first dose, and increasing after the second dose administered on Day 21 (FIGS. 11-12). Neutralizing antibodies were also determined with the wild type virus using the plaque reduction assay, as described previously and showed a similar response across treatment groups (FIG. 13).

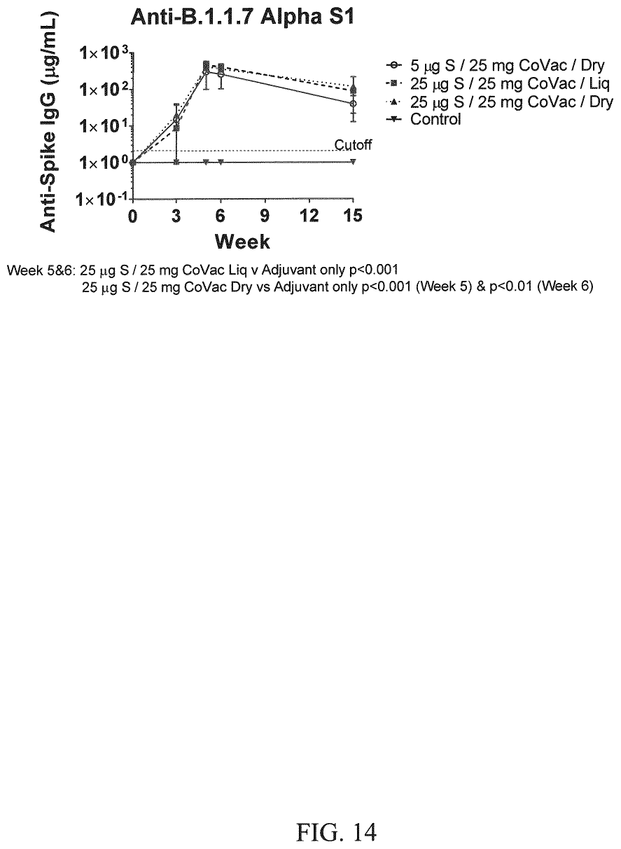

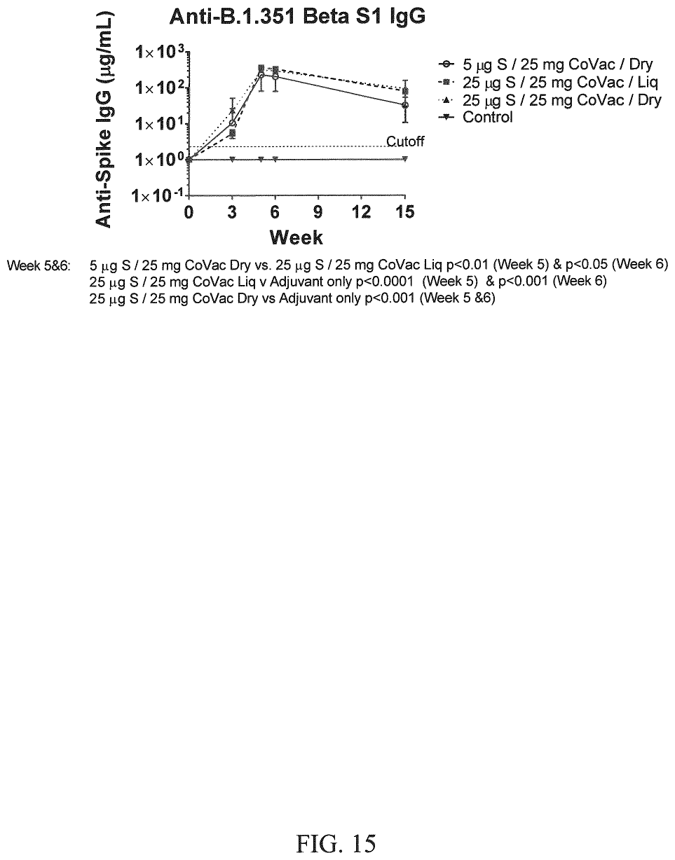

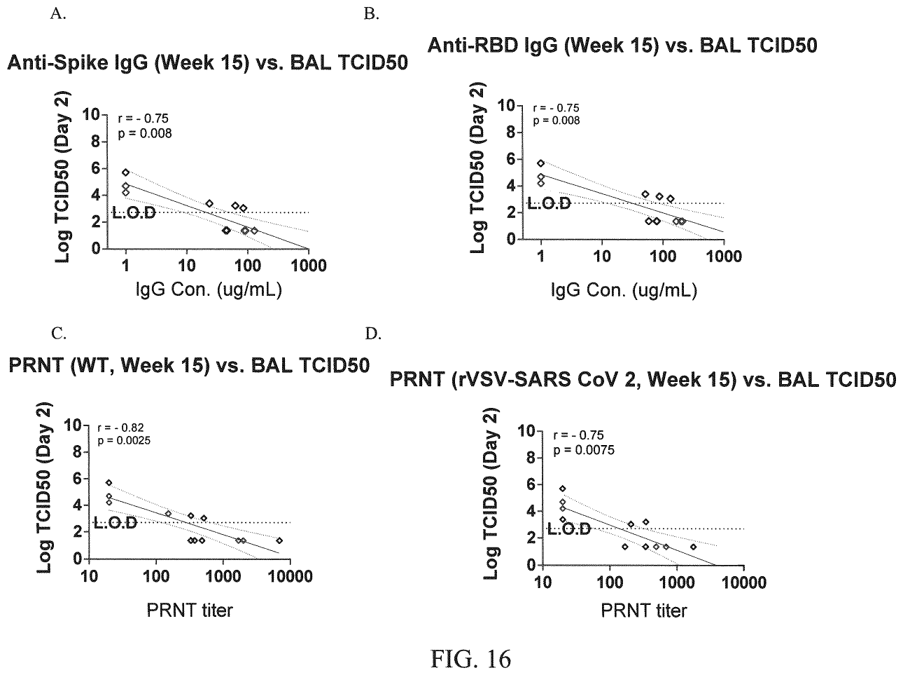

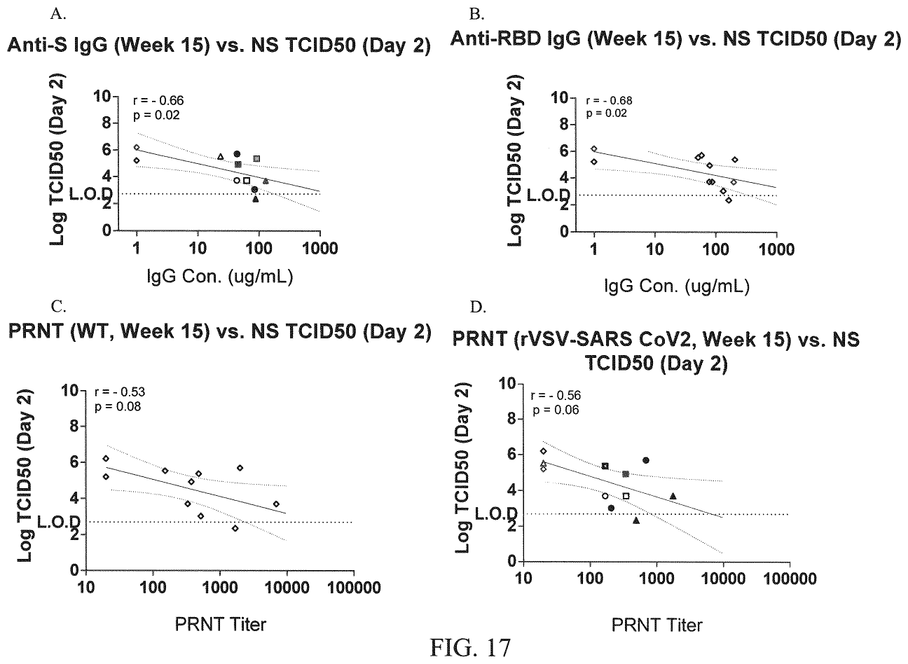

Evaluation of the neutralizing antibody response to COVID-19 variants (alpha—FIG. 14; beta—FIG. 15) was also strong. At Week 15 (FIG. 10), animals were challenged with P.1 Gamma Variant of Concern. There was a significant correlation between vaccination status and the reduction in viral load in the bronchoalveolar lavage (BAL) fluid (determined by the median tissue culture infectious dose; TCID50) (FIG. 16), with lesser responses in the nasal cavity, as expected (FIG. 17).

### Discussion

An ideal COVID-19 vaccine is expected to induce both humoral and cellular immunity, high titers of neutralizing antibodies and Thl-biased response to reduce potential risk of vaccine-associated enhancement of disease [5, 40, 41]. The CoVaccine HT™ adjuvant is demonstrated to be a superior adjuvant for generating both humoral and cell mediated immunity. Combination with the S1 subunit of the Spike proteins of SARS-CoV-2 demonstrated strong immunogenicity.

Using a well-established insect cell expression system, the present invention also provides two versions of the SARS-CoV-2 S protein ectodomain and formulates them with a potent adjuvant, CoVaccine HTTM. The vaccine candidates elicit both neutralizing antibody and cellular immunity with a balanced Thl/Th2 response in an outbred mouse model. The results obtained in this model may thus inform future vaccine development in animal models more closely related to humans [42]. This supports further preclinical and clinical development of CoVaccine HT™ adjuvanted SARS-CoV-2 vaccine to mitigate the ongoing COVID-19 pandemic.

The S protein of SARS-CoV-2 contains a total of 1,273 amino acids and two major domains (S1 and S2) with distinct structures and functions. Previous preclinical studies of SARS-CoV and MERS-CoV vaccines have demonstrated that the S protein plays a key role in induction of neutralizing antibody and T cell responses as well as protective immunity [16-18]. Stabilization of S proteins in the prefusion trimeric conformation results in increased expression, conformational homogeneity, and production of potent neutralizing antibody responses [18, 27]. Current SARS-CoV-2 vaccines under development use either RBD or full-length S protein with or without modifications for stabilization of prefusion conformation as the major antigen targets. Although RBD is a primary target for potent neutralizing antibodies, it lacks other neutralizing epitopes present on full-length S. This might suggest that full-length S-based vaccines would broaden the neutralizing repertoire and reduce the potential of viral escape from host immunity.

The present invention further describes trimeric S ectodomains in which the furin cleavage site was mutated (SdTM), which were further stabilized in the prefusion S form by removing the S2′ protease cleavage site and introducing two proline substitutions (SdTM2P). The introduction of two prolines in the S2 subunit resulted in significantly greater production yield (˜3-fold increase) in Drosophila S2 cells (data not shown). Vaccination with adjuvanted SdTM or SdTM2P elicits comparable levels of IgG antibody and IFN-γ cellular responses; however, the SdTM2P vaccine generated a slightly higher level of neutralizing antibodies than SdTM, which was also reported in studies of SARS-CoV, MERS-CoV, and other SARS-CoV-2 vaccines [18, 24]. Although more investigation is required to understand whether stabilization of prefusion S ectodomain enhances immunogenicity, the production of stabilized prefusion antigens represents a promising strategy for COVID-19 vaccine design.

CoVaccine HT™ is a novel adjuvant that consists of a sucrose fatty acid sulfate ester (SFASE) immobilized on the oil droplets of a submicrometer emulsion of squalane in water (oil-in-water emulsion) [43]. It has been used for influenza virus and malaria vaccines and shown to enhance humoral and cellular protective immunity, in particular antibody response [44-48]. Use of CoVaccine HT™ with SdTM and SdTM2P yielded significantly enhanced total IgG and neutralizing antibody responses after both the first and second dose as compared to protein alone which reached similar IgG concentrations after two doses as a single dose of the adjuvanted formulations. Antibody levels (total IgG or neutralizing) did not decrease with a decreased dose of adjuvant, instead a pronounced increase in neutralizing antibody titers were observed, indicating further opportunity for formulation optimization.

In addition to improved kinetics, the addition of CoVaccine HT™ modulated the humoral response more towards Th1 type relative to protein alone, as indicated by higher levels of IgG2a and IgG2b. Antigen-specific splenocyte restimulation was more variable, but also increased with the use of adjuvant, particularly after two doses, and were not strongly dependent on adjuvant concentration. These robust responses in outbred mice observed with relatively low antigen and adjuvant doses are particularly encouraging. The similarity of the COVID-19 candidate formulation to prior thermostabilization efforts provides the potential for it to also be similarly thermostabilized in a single vial format. This would allow easier vaccine stockpiling and distribution in regions of the world incapable of maintaining cold-chain logistics necessary for transporting and storing vaccines from other platforms.

The urgent need to rapidly vaccinate the human population worldwide both to slow the increase in fatalities and to prevent the emergence of escape mutations sharply highlights the limitations of vaccines requiring stringent cold chains as even under the best conditions, doses are lost due to inadequate storage or handling. The use of protein subunit vaccines not only offers additional vaccines to be used in parallel to rapidly immunize the global population, but also offers an easier opportunity to develop thermostabilized vaccines which would simplify rapid and far-flung delivery. Using recombinant spike protein without the transmembrane domain and with pre-fusion complex stabilization, the present invention demonstrates robust immune responses with the CoVaccine HT™ adjuvant in an outbred mouse model. This immune response is observed within 7 days of the first dose and peaks within 14 days after the second dose, showing robust humoral and cell mediated immunity. The immune response was potent with as little as 2.5 μg of protein and 0.3 mg of adjuvant, indicating an economical dose-sparing format should be feasible. NHP studies yielded a robust antibody response after the first vaccination, increasing after the second vaccination, with as little as 5 μg immunogen and in the presence of both liquid and lyophilized adjuvant (5 mg). These results suggest that a single-vial, ambiently-stored vaccine formulation should be feasible.

As used in this specification and in the appended claims, the singular forms include the plural forms. For example the terms “a,” “an,” and “the” include plural references unless the content clearly dictates otherwise. Additionally, the term “at least” preceding a series of elements is to be understood as referring to every element in the series. The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein. In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. Those skilled in the art will recognize, or will be able to ascertain using no more than routine experimentation, many equivalents to the specific embodiments of the invention described. Such equivalents are intended to be encompassed by the following claims.

### REFERENCES

[1] Johns Hopkins University Coronavirus Resource Center. COVID-19 case tracker.
[2] World Health Organization. Draft landscape of COVID-19 candidate vaccines. 29 December 2020.
[3] Pfizer-BioNTech COVID-19 Vaccine. https://www.fda.gov/emergency-preparedness-and-response/coronavirus-di sease-2019-covid-19/pfizer-biontech-covid-19-vaccine. : Food and Drug Administration 2021.
[4] Moderna CO VID-19 Vaccine. https ://www.fda.gov/emergency-preparedness-and-response/coronavirus-di sease-2019-covid-19/moderna-covid-19-vaccine.: U.S, Food and Drug Adminitration; 2021.
[5] Krammer F. SARS-CoV-2 vaccines in development. Nature. 2020;586:516-27.
[6] Medina L O, To A, Lieberman M M, Wong T A S, Namekar M, Nakano E, et al. A Recombinant Subunit Based Zika Virus Vaccine Is Efficacious in Non-human Primates. Frontiers in immunology. 2018;9:2464.
[7] To A, Medina L O, Mfuh K O, Lieberman M M, Wong T A S, Namekar M, et al. Recombinant Zika Virus Subunits Are Immunogenic and Efficacious in Mice. mSphere. 2018;3.
[8] Lehrer A T, Wong T S, Lieberman M M, Humphreys T, Clements D E, Bakken R R, et al. Recombinant proteins of Zaire ebolavirus induce potent humoral and cellular immune responses and protect against live virus infection in mice. Vaccine. 2018;36:3090-100.
[9] Lehrer A T, Wong T S, Lieberman M M, Johns L, Medina L, Feldmann F, et al. Recombinant subunit vaccines protect guinea pigs from lethal Ebola virus challenge. Vaccine. 2019;37:6942-50.
[10] Chisholm C F, Kang T J, Dong M, Lewis K, Namekar M, Lehrer A T, et al. Thermostable Ebola virus vaccine formulations lyophilized in the presence of aluminum hydroxide. European journal of pharmaceutics and biopharmaceutics: official journal of Arbeitsgemeinschaft fur Pharmazeutische Verfahrenstechnik eV. 2019;136:213-20.
[11] Preston K B, Monticello C R, Wong T A S, To A, Donini O, Lehrer AT, et al. Preservation of Quaternary Structure in Thermostable, Lyophilized Filovirus Glycoprotein Vaccines: A Search for Stability-Indicating Assays. Journal of pharmaceutical sciences. 2020;109:3716-27.
[12] Westfall J, Yates J L, Van Slyke G, Ehrbar D, Measey T, Straube R, et al. Thermal stability and epitope integrity of a lyophilized ricin toxin subunit vaccine. Vaccine. 2018;36:5967-76.
[13] Tortorici M A, Veesler D. Structural insights into coronavirus entry. Advances in virus research. 2019;105:93-116.
[14] Yong C Y, Ong H K, Yeap S K, Ho K L, Tan W S. Recent Advances in the Vaccine Development Against Middle East Respiratory Syndrome-Coronavirus. Frontiers in microbiology. 2019;10:1781.
[15] Graham R L, Donaldson E F, Baric R S. A decade after SARS: strategies for controlling emerging coronaviruses. Nature reviews Microbiology. 2013;11:836-48.
[16] Pallesen J, Wang N, Corbett K S, Wrapp D, Kirchdoerfer R N, Turner H L, et al. Immunogenicity and structures of a rationally designed prefusion MERS-CoV spike antigen. Proceedings of the National Academy of Sciences of the United States of America. 2017; 114 :E7348-e57.
[17] Okba N M A, Muller M A, Li W, Wang C, GeurtsvanKessel C H, Corman V M, et al. Severe Acute Respiratory Syndrome Coronavirus 2-Specific Antibody Responses in Coronavirus Disease Patients. Emerging infectious diseases. 2020;26:1478-88.
[18] Wajnberg A, Amanat F, Firpo A, Altman D R, Bailey M J, Mansour M, et al. Robust neutralizing antibodies to SARS-CoV-2 infection persist for months. Science (New York, NY). 2020;370:1227-30.
[19] Zhou P, Yang X L, Wang X G, Hu B, Zhang L, Zhang W, et al. A pneumonia outbreak associated with a new coronavirus of probable bat origin. Nature. 2020;579:270-3.
[20] Grifoni A, Weiskopf D, Ramirez S I, Mateus J, Dan J M, Moderbacher C R, et al. Targets of T Cell Responses to SARS-CoV-2 Coronavirus in Humans with COVID-19 Disease and Unexposed Individuals. Cell. 2020;181:1489-501.e15.
[21] Amanat F, Strohmeier S, Rathnasinghe R, Schotsaert M, Coughlan L, Garcia-Sastre A, et al. Introduction of two prolines and removal of the polybasic cleavage site leads to optimal efficacy of a recombinant spike based SARS-CoV-2 vaccine in the mouse model. bioRxiv : the preprint server for biology. 2020.
[22] Mercado N B, Zahn R, Wegmann F, Loos C, Chandrashekar A, Yu J, et al. Single-shot Ad26 vaccine protects against SARS-CoV-2 in rhesus macaques. Nature. 2020;586:583-8.
[23] Keech C, Albert G, Cho I, Robertson A, Reed P, Neal S, et al. Phase 1-2 Trial of a SARS-CoV-2 Recombinant Spike Protein Nanoparticle Vaccine. The New England journal of medicine. 2020;383 :2320-32.
[24] Corbett K S, Edwards D K, Leist S R, Abiona O M, Boyoglu-Barnum S, Gillespie RA, et al. SARS-CoV-2 mRNA vaccine design enabled by prototype pathogen preparedness. Nature. 2020;586:567-71.
[25] Hsieh C L, Goldsmith J A, Schaub J M, DiVenere A M, Kuo H C, Javanmardi K, et al. Structure-based design of prefusion-stabilized SARS-CoV-2 spikes. Science (New York, NY). 2020;369:1501-5.
[26] Graham B S, Gilman M S A, McLellan J S. Structure-Based Vaccine Antigen Design. Annual review of medicine. 2019;70:91-104.
[27] Haun B K, Lai C Y, Williams C A, Wong T A S, Lieberman M M, Pessaint L, et al. CoVaccine HT™ Adjuvant Potentiates Robust Immune Responses to Recombinant SARS-CoV-2 Spike S1 Immunization. Frontiers in immunology. 2020;11:599587.
[28] Namekar M, Kumar M, O'Connell M, Nerurkar V R. Effect of serum heat-inactivation and dilution on detection of anti-WNV antibodies in mice by West Nile virus E-protein microsphere immunoassay. PloS one. 2012;7:e45851.
[29] Schmidt F, Weisblum Y, Muecksch F, Hoffmann H H, Michailidis E, Lorenzi J C C, et al. Measuring SARS-CoV-2 neutralizing antibody activity using pseudotyped and chimeric viruses. The Journal of experimental medicine. 2020;217.
[30] Case J B, Rothlauf P W, Chen R E, Liu Z, Zhao H, Kim A S, et al. Neutralizing Antibody and Soluble ACE2 Inhibition of a Replication-Competent VSV-SARS-CoV-2 and a Clinical Isolate of SARS-CoV-2. Cell host & microbe. 2020;28:475-85.e5.
[31] Kim H W, Canchola J G, Brandt C D, Pyles G, Chanock R M, Jensen K, et al. Respiratory syncytial virus disease in infants despite prior administration of antigenic inactivated vaccine. American journal of epidemiology. 1969;89:422-34.
[32] Chin J, Magoffin R L, Shearer L A, Schieble J H, Lennette E H. Field evaluation of a respiratory syncytial virus vaccine and a trivalent parainfluenza virus vaccine in a pediatric population. American journal of epidemiology. 1969;89:449-63.
[33] Fulginiti V A, Eller J J, Downie A W, Kempe C H. Altered reactivity to measles virus. Atypical measles in children previously immunized with inactivated measles virus vaccines. Jama. 1967;202:1075-80.
[34] Browne S K, Beeler J A, Roberts J N. Summary of the Vaccines and Related Biological Products Advisory Committee meeting held to consider evaluation of vaccine candidates for the prevention of respiratory syncytial virus disease in RSV-naive infants. Vaccine. 2020;38:101-6.
[35] Czub M, Weingartl H, Czub S, He R, Cao J. Evaluation of modified vaccinia virus Ankara based recombinant SARS vaccine in ferrets. Vaccine. 2005;23:2273-9.
[36] Bolles M, Deming D, Long K, Agnihothram S, Whitmore A, Ferris M, et al. A double-inactivated severe acute respiratory syndrome coronavirus vaccine provides incomplete protection in mice and induces increased eosinophilic proinflammatory pulmonary response upon challenge. Journal of virology. 2011;85:12201-15.
[37] Tseng C T, Sbrana E, Iwata-Yoshikawa N, Newman P C, Garron T, Atmar RL, et al. Immunization with SARS coronavirus vaccines leads to pulmonary immunopathology on challenge with the SARS virus. PloS one. 2012;7:e35421.
[38] Graham BS. Rapid COVID-19 vaccine development. Science (New York, NY). 2020;368:945-6.
[39] Jeyanathan M, Afkhami S, Smaill F, Miller M S, Lichty B D, Xing Z. Immunological considerations for COVID-19 vaccine strategies. Nature reviews Immunology. 2020;20:615-32.
[40] Tuttle A H, Philip V M, Chesler E J, Mogil J S. Author Correction: Comparing phenotypic variation between inbred and outbred mice. Nature methods. 2020;17:947.
[41] Blom A G, Hilgers L A. Sucrose fatty acid sulphate esters as novel vaccine adjuvants: effect of the chemical composition. Vaccine. 2004;23:743-54.
[42] Bodewes R, Kreijtz J H, van Amerongen G, Geelhoed-Mieras M M, Verburgh R J, Heldens J G, et al. A single immunization with CoVaccine HT-adjuvanted H5N1 influenza virus vaccine induces protective cellular and humoral immune responses in ferrets. Journal of virology. 2010;84:7943-52.
[43] Heldens J G, Glansbeek H L, Hilgers L A, Haenen B, Stittelaar K J, Osterhaus A D, et al. Feasibility of single-shot H5N1 influenza vaccine in ferrets, macaques and rabbits. Vaccine. 2010;28:8125-31.
[44] Mandi Abdel Hamid M, Remarque E J, van Duivenvoorde L M, van der Werff N, Walraven V, Faber B W, et al. Vaccination with Plasmodium knowlesi AMA1 formulated in the novel adjuvant co-vaccine HT™ protects against blood-stage challenge in rhesus macaques. PloS one. 2011;6:e20547.
[45] Kusi K A, Remarque E J, Riasat V, Walraven V, Thomas A W, Faber B W, et al. Safety and immunogenicity of multi-antigen AMA1-based vaccines formulated with CoVaccine HT™ and Montanide ISA 51 in rhesus macaques. Malaria journal. 2011;10:182.
[46] Lakhan N, Stevens N E, Diener K R, Hayball J D. CoVaccine HT™ adjuvant is superior to Freund's adjuvants in eliciting antibodies against the endogenous alarmin HMGB1. Journal of immunological methods. 2016;439:37-43.

What is claimed is:

1. A method of inducing protective immunity to a coronavirus within an individual, comprising providing a stable immunogenic composition capable of eliciting a robust and durable immune response to the coronavirus, wherein the composition comprises a protein subunit comprising a recombinant protein specific to the coronavirus and at least one adjuvant; and administering an effective amount of the composition to the individual.

2. The method of claim 1, wherein the recombinant protein specific to the coronavirus is expressed using insect cells.

3. The method of claim 1, wherein the protein subunit is a spike protein from the coronavirus.

4. The method of claim 3, wherein the spike protein is further purified using immunoaffinity purification. The method of claim 1, wherein the protein subunit is present in the composition from about 1 μg to about 25 μg.

6. The method of claim 1, wherein the at least one adjuvant is a sucrose fatty acid sulphate ester.

7. The method of claim 1, wherein the at least one adjuvant is present in the composition from about 0.3 mg to about 10 mg.

8. The method of claim 1, wherein the protein subunit and the at least one adjuvant are each thermostabilized separately before being combined in the composition.

9. The method of claim 1, wherein the protein subunit and the at least one adjuvant are thermostabilized together before being combined in the composition.

10. A method of adjuvanting subunit vaccines, comprising providing an effective amount of an adjuvant, providing a protein subunit and combining the adjuvant and the protein subunit to create a stable immunogenic composition capable of eliciting a robust and durable immune response to a coronavirus, wherein the adjuvant is a sucrose fatty acid sulphate ester at a concentration selected from the group consisting of between 0.3 and 1 mg, 1 mg and 5 mg and 5 mg and 10 mg.

11. The method of claim 10, wherein the protein subunit is specific to the coronavirus and is expressed using insect cells.

12. The method of claim 10, wherein the protein subunit is a spike protein from the coronavirus.

13. The method of claim 12, wherein the spike protein is further purified using immunoaffinity purification.

14. The method of claim 10, wherein the protein subunit is present in the composition from about 1 μg to about 25 μg.

15. The method of claim 10, wherein the protein subunit and the adjuvant are each thermostabilized separately before being combined in the composition.

16. The method of claim 1, wherein the protein subunit and the at least one adjuvant are thermostabilized together before being combined in the composition.
